(19) United States
(12) Patent Application Publication
(10) Pub. No.: US 2021/0201701 A1
(43) Pub. Date: Jul. 1, 2021

(54) SYSTEMS AND METHODS FOR MEDICAL DIAGNOSIS TRAINING

(71) Applicant: SHANGHAI UNITED IMAGING INTELLIGENCE CO., LTD., Shanghai (CN)

(72) Inventors: Rui LI, Shanghai (CN); Hong SHEN, Shanghai (CN)

(73) Assignee: SHANGHAI UNITED IMAGING INTELLIGENCE CO., LTD., Shanghai (CN)

(21) Appl. No.: 17/133,578

(22) Filed: Dec. 23, 2020

(30) Foreign Application Priority Data
Dec. 25, 2019 (CN) 201911359141.1

(51) Int. Cl. G09B 23/28; A61B 90/00; G09B 5/02

(52) U.S. Cl. CPC G09B 23/286; G09B 5/02; A61B 90/00

(57) ABSTRACT

A system for medical diagnosis training is provided. The system may receive, from a user terminal, a training request inputted by a user. In response to the training request, the system may obtain a medical image for training the user and a reference diagnostic result with respect to the medical image, and transmit the medical image to the user terminal. The system may receive, from the user terminal, a diagnostic result with respect to the medical image inputted by the user. The system may further generate an evaluation result of the diagnostic result based on the reference diagnostic result.

### CROSS-REFERENCE TO RELATED APPLICATIONS

This application claims priority of Chinese Patent Application No. 201911359141.1, filed on Dec. 25, 2019, the contents of which are hereby incorporated by reference.

### TECHNICAL FIELD

The present disclosure generally relates to medical diagnosis training, and more particularly, relates to systems and methods for evaluating a diagnostic result with respect to a medical image in medical diagnostic training.

### BACKGROUND

Medical imaging techniques are widely used in medical diagnosis to acquire a medical image of a subject (e.g., a patient). Medical personnel (e.g., a doctor, a nurse, a physician) often needs to provide a diagnostic result with respect to the subject based on the medical image. The quality of the diagnostic result may affect a subsequent treatment of the subject. For example, the quality of the diagnostic result may affect the selection of a treatment principle and/or the generation of a treatment plan for the subject. However, it is hard to provide an accurate diagnostic result, and the quality of the diagnostic result may be associated with the experience and professional level of the medical personnel. On the other hand, compared with a large hospital (e.g., a three-A hospital in China), a small hospital (e.g., a township hospital) may have a limited count of medical images for training medical personnel, and the medical personnel in the small hospital may lack medical practice. Some medical personnel, e.g., who have limited experience and/or work in a small hospital, may need to receive training to improve their diagnostic capacity with respect to medical images. Thus, it is desirable to develop systems and methods for medical diagnosis training.

### SUMMARY

According to one aspect of the present disclosure, a system for medical diagnosis training is provided. The system may include at least one storage device including a set of instructions, and at least one processor configured to communicate with the at least one storage device. When executing the set of instructions, the at least one processor may be configured to direct the system to perform operations. The at least one processor may be configured to direct the system to receive, from a user terminal, a training request inputted by a user. In response to the training request, the at least one processor may be configured to direct the system to obtain a medical image for training the user and a reference diagnostic result with respect to the medical image. The at least one processor may be also configured to direct the system to transmit the medical image to the user terminal, and receive a diagnostic result with respect to the medical image inputted by the user from the user terminal. The at least one processor may be further configured to direct the system to generate an evaluation result of the diagnostic result based on the reference diagnostic result.

In some embodiments, the evaluation result of the diagnostic result may include at least one of a value of a first evaluation index relating to a description order of the diagnostic result, a value of a second evaluation index relating to a content integrity of the diagnostic result, a value of a third evaluation index relating to an accuracy of the diagnostic result, or a value of a fourth evaluation index relating to a similarity between the diagnostic result and the reference diagnostic result.

In some embodiments, the at least one processor may be configured to direct the system to generate a first vector representing the diagnostic result, generate a second vector representing the reference diagnostic result, and generate the evaluation result of the diagnostic result based on the first vector and the second vector.

In some embodiments, the at least one processor may be configured to direct the system to determine at least one first structured sequence based on the first vector, and determine at least one second structured sequence based on the second vector. Each of the at least one first structured sequence may include a first anatomical structure, a first feature of the first anatomical structure, and a first feature value of the first feature recorded in the diagnostic result. Each of the at least one second structured sequence may include a second anatomical structure, a second feature of the second anatomical structure, and a second feature value of the second feature recorded in the reference diagnostic result. The at least one processor may be further configured to direct the system to generate the evaluation result of the diagnostic result based on the at least one first structured sequence and the second structured sequence.

In some embodiments, the at least one processor may be configured to direct the system to generate at least one normalized first structured sequence by normalizing the at least one first structured sequence, generate at least one normalized second structured sequence by normalizing the at least one second structured sequence, and generate the evaluation result of the diagnostic result based on the at least one normalized first structured sequence and the at least one normalized second structured sequence.

In some embodiments, the at least one processor may be configured to direct the system to determine first ordering information relating to the diagnostic result, and determine second ordering information relating to the reference diagnostic result. The first ordering information may reflect an order of appearance of the at least one first anatomical structure of the at least one first structured sequence in the diagnostic result. The second ordering information may reflect an order of appearance of the at least one second anatomical structure of the at least one second structured sequence in the reference diagnostic result. The at least one processor may be further configured to direct the system to determine a value of an evaluation index relating to a description order of the diagnostic result based on the first ordering information and the second ordering information.

In some embodiments, the first ordering information may further reflect an order of appearance of the at least one first feature of the at least one first anatomical structure in the diagnostic result, and the second ordering information may further reflect an order of appearance of the at least one second feature of the at least one second anatomical structure in the reference diagnostic result.

In some embodiments, the at least one processor may be configured to direct the system to generate a first comparison result by comparing the at least one first anatomical structure of the at least one first structured sequence and the at least one second anatomical structure of the at least one second structured sequence, and generate a second comparison result by comparing the at least one first feature of the at least one first structured sequence and the at least one second feature of the at least one second structured sequence. The at least one processor may be further configured to direct the system to determine a value of an evaluation index relating to a content integrity of the diagnostic result based on the first compassion result and the second comparison result.

In some embodiments, the at least one processor may be configured to direct the system to determine at least one first characteristic value of at least one first lesion specified in the diagnostic result based on the first vector, and determine at least one second characteristic value of at least one second lesion specified in the reference diagnostic result based on the second vector. The at least one processor may be configured to direct the system to determine a value of an evaluation index relating to an accuracy of the diagnostic result based on the at least one first characteristic value and the at least one second characteristic value.

In some embodiments, the at least one processor may be configured to direct the system to determine at least one first target vector based on the first vector, and determine at least one second target vector based on the second vector. Each of the at least one first target vector may represent a first element of the diagnostic result. Each of the at least one second target vector may represent a second element of the reference diagnostic result. The at least one processor may be configured to direct the system to determine a value of an evaluation index relating to a similarity between the diagnostic result and the reference diagnostic result based on the at least one first target vector and the at least one second target vector.

In some embodiments, the at least one processor may be configured to direct the system to obtain a user profile of the user, and determine a plurality of candidate medical images for training the user based on the user profile of the user. The at least one processor may be configured to direct the system to select the medical image from the plurality of candidate medical images based on a recommendation rule.

In some embodiments, the user profile may include at least one of user preference information, information relating to one or more medical images that have been used to train the user, or one or more historical diagnostic results inputted by the user.

In some embodiments, the plurality of candidate medical images may be determined based on the user profile of the user using a knowledge graph technique.

In some embodiments, the at least one processor may be configured to direct the system to generate the reference diagnostic result by evaluating the medical image using a diagnostic result generation model. The diagnostic result generation model may be generated using an artificial intelligence technique.

According to another aspect of the present disclosure, a method for medical diagnosis training is provided. The method may include receiving, from a user terminal, a training request inputted by a user. The method may include, in response to the training request, obtaining a medical image for training the user and a reference diagnostic result with respect to the medical image. The method may also include transmitting the medical image to the user terminal. The method may also include receiving, from the user terminal, a diagnostic result with respect to the medical image inputted by the user. The method may further include generating an evaluation result of the diagnostic result based on the reference diagnostic result.

According to another aspect of the present disclosure, a non-transitory readable medium including at least one set of instructions is provided. When executed by at least one processor of a system for medical diagnosis training, the at least one set of instructions may direct the at least one processor to perform a method. The method may include receiving, from a user terminal, a training request inputted by a user. The method may include, in response to the training request, obtaining a medical image for training the user and a reference diagnostic result with respect to the medical image. The method may also include transmitting the medical image to the user terminal. The method may also include receiving, from the user terminal, a diagnostic result with respect to the medical image inputted by the user. The method may further include generating an evaluation result of the diagnostic result based on the reference diagnostic result.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary training system for medical diagnosis training according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device according to some embodiments of the present disclosure;

FIG. 4 is a block diagram illustrating an exemplary second server according to some embodiments of the present disclosure;

FIG. 5 is a flowchart illustrating an exemplary process for medical diagnosis training according to some embodiments of the present disclosure;

FIG. 6 is a schematic diagram illustrating an exemplary process for obtaining a medical image for training a user according to some embodiments of the present disclosure;

FIG. 7 is a schematic diagram illustrating an exemplary process for generating an evaluation result of a diagnostic result based on a reference diagnostic result according to some embodiments of the present disclosure;

FIG. 8 is a flowchart illustrating an exemplary process for determining a value of a first evaluation index relating to a description order of a diagnostic result according to some embodiments of the present disclosure;

FIG. 9 is a flowchart illustrating an exemplary process for determining a value of a second evaluation index relating to a content integrity of a diagnostic result according to some embodiments of the present disclosure;

FIG. 10 is a flowchart illustrating an exemplary process for determining a value of a third evaluation index relating to an accuracy of a diagnostic result according to some embodiments of the present disclosure;

FIG. 11 is a flowchart illustrating an exemplary process for determining a value of a fourth evaluation index relating to a similarity between a diagnostic result and a reference diagnostic result according to some embodiments of the present disclosure;

FIG. 12 is a schematic diagram illustrating an exemplary process for generating an evaluation result of a diagnostic result based on a reference diagnostic result according to some embodiments of the present disclosure;

FIG. 13 is a schematic diagram illustrating an exemplary process for generating an evaluation result of a diagnostic result based on a reference diagnostic result according to some embodiments of the present disclosure;

FIG. 14 is a schematic diagram illustrating an exemplary process for generating an evaluation result of a diagnostic result based on a reference diagnostic result according to some embodiments of the present disclosure;

FIG. 15 is a schematic diagram illustrating an exemplary process for generating an evaluation result of a diagnostic result based on a reference diagnostic result according to some embodiments of the present disclosure;

FIG. 16 is a schematic diagram illustrating an exemplary process for selecting a medical image for training a user according to some embodiments of the present disclosure; and

FIG. 17 is a schematic diagram illustrating an exemplary user interface of a user terminal according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

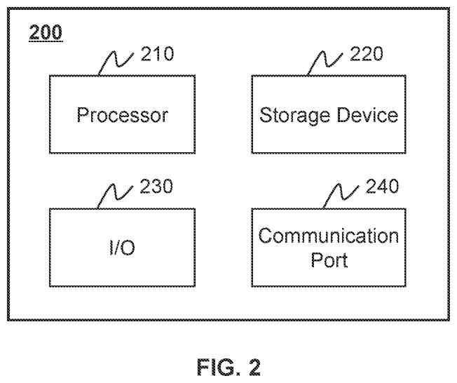

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Provided herein are systems and methods for medical diagnosis training based on medical images. The medical images may be generated via a non-invasive biomedical imaging system. In some embodiments, the system may include a single modality imaging system and/or a multi-modality imaging system. The single modality imaging system may include, for example, an ultrasound imaging system, an X-ray imaging system, an computed tomography (CT) system, a magnetic resonance imaging (MRI) system, an ultrasonography system, a positron emission tomography (PET) system, an optical coherence tomography (OCT) imaging system, an ultrasound (US) imaging system, an intravascular ultrasound (IVUS) imaging system, a near infrared spectroscopy (NIRS) imaging system, a far infrared (FIR) imaging system, or the like, or any combination thereof. The multi-modality imaging system may include, for example, an X-ray imaging-magnetic resonance imaging (X-ray-MRI) system, a positron emission tomography-X-ray imaging (PET-X-ray) system, a single photon emission computed tomography-magnetic resonance imaging (SPECT-MRI) system, a positron emission tomography-computed tomography (PET-CT) system, a C-arm system, a digital subtraction angiography-magnetic resonance imaging (DSA-MRI) system, etc. It should be noted that the imaging system described below is merely provided for illustration purposes, and not intended to limit the scope of the present disclosure.

The term “imaging modality” or “modality” as used herein broadly refers to an imaging method or technology that gathers, generates, processes, and/or analyzes imaging information of a subject. The subject may include a biological subject and/or a non-biological subject. The biological subject may be a human being, an animal, a plant, or a portion thereof (e.g., a cell, a tissue, an organ, etc.). In some embodiments, the subject may be a man-made composition of organic and/or inorganic matters that are with or without life.

Medical diagnosis is often performed based on a medical image. In order to improve the diagnostic accuracy, medical personnel may need to receive medical diagnostic training to improve their ability to perform medical diagnosis based on a medical image. For example, some large hospitals may organize training activities regularly to train medical personnel on-site. As another example, medical personnel may be trained through a remote video training, for example, by watching a teaching video (e.g., a recorded video, a live video). However, theses conventional training approaches normally use same or similar training material to train medical personnel with different professional levels, which are lack pertinence and have a poor training result. In addition, an expert who teaches the medical personnel may need to prepare teaching material (e.g., a teaching video) in advance, which costs a lot of time and effort.

An aspect of the present disclosure relates to systems and methods for medical diagnosis training. The systems and methods may receive a training request inputted by a user from a user terminal. In response to the training request, the systems and methods may obtain a medical image for training the user and a reference diagnostic result with respect to the medical image. The systems and methods may transmit the medical image to the user terminal. The systems and methods may also receive a diagnostic result with respect to the medical image inputted by the user from the user terminal. The systems and methods may generate an evaluation result of the diagnostic result based on the reference diagnostic result.

For example, the evaluation result may include values of various evaluation indexes relating to different features of the diagnostic result. Merely by way of example, the evaluation result may be determined based on a first evaluation index relating to a description order of the diagnostic result, a second evaluation index relating to a content integrity of the diagnostic result, a third evaluation index relating to an accuracy of the diagnostic result, and a fourth evaluation index relating to a similarity between the diagnostic result and the reference diagnostic result. The various evaluation indexes may be used to comprehensively evaluate the diagnostic result from different perspectives.

According to some embodiments of the present disclosure, one or more first servers may be deployed in one or more hospitals having a certain scale to collect training materials generated in the hospital(s). A second server may be configured to manage training materials received from the first server(s) and implement medical diagnosis training processes disclosed herein. For example, a user (e.g., a doctor of a downtown hospital) may use his/her user terminal to transmit a training request to the second server. The second server may determine a suitable medical image for training the user (e.g., a medical image relating to a disease that the user is interested in), and evaluate a diagnostic result with respect to the medical image inputted by the user. In this way, the systems and the methods may provide remote and automatic medical diagnosis training for the user, which may be implemented without or with reduced help of a senior doctor. In addition, the user may get access to plenty of medical images via the second server in real-time, thereby providing more training resources for the user and improving the training effect. Additionally or alternatively, in some embodiments of the present disclosure, the reference diagnostic result of the medical image may be generated using an artificial intelligence (AI) technique. By using the reference diagnostic result that is determined using the AI technique, the evaluation accuracy of the diagnostic result may be improved, and the training efficiency may be improved by reducing, e.g., the human and/or time resources for preparing training material.

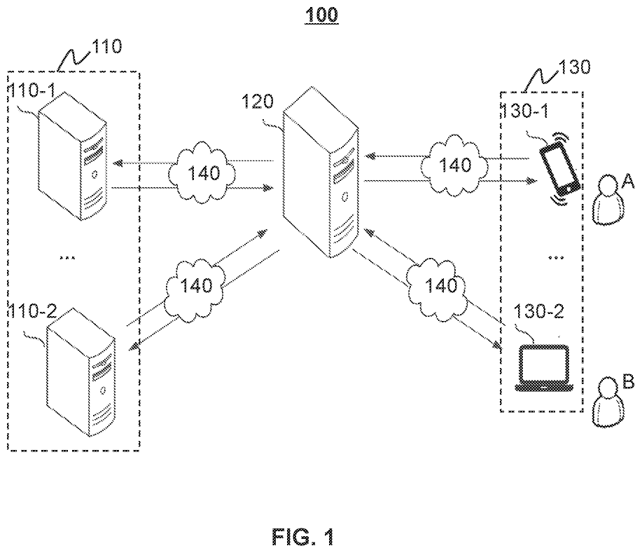

FIG. 1 is a schematic diagram illustrating an exemplary training system 100 for medical diagnosis training according to some embodiments of the present disclosure. As shown in FIG. 1, the training system 100 may include one or more first servers 110 (e.g., a first server 110-1, a first server 110-2), a second server 120, one or more terminals 130, and a network 140. A terminal 130 may be operated by a user (e.g., a user A, a user B). As used herein, a user refers to any entity (e.g., medical personnel, a medical student, an AI device for medical diagnosis) that needs to receive medical diagnosis training. In some embodiments, the first server(s) 110, the second server 120, and the terminal(s) 130 may be connected to and/or communicate with each other via a wireless connection, a wired connection, or a combination thereof.

The first server(s) 110 may be configured to obtain and/or generate training material used in medical diagnosis training. The training material may relate to one or more medical images of one or more subjects (e.g., a patient). Exemplary training material relating to a medical image of a subject may include the medical image itself, information relating to the medical image, information relating to the subject, a reference diagnostic result with respect to the medical image, or the like, or any combination thereof. Exemplary information relating to the medical image may include information relating to an imaging device or an imaging modality that generates the medical image, an imaging condition under which the medical image is generated, a difficulty level of the medical image, a hospital department corresponding to the medical image, a count of times that the medical image is marked by users of the training system 100, a count of times that users of the training system 100 provide a false diagnostic result with respect to the medical image, or the like, or any combination thereof. Exemplary information relating to the subject may include a name, a gender, an age, a phone number, an identity (ID) card number, a medical record (such as a treatment history, a smoking history, and/or a drinking history), a major disease, a secondary disease, an imaging part, other medical images, or the like, or any combination thereof, of the subject. The reference diagnostic result with respect to the medical image may be provided or confirmed by senior medical personnel. Alternatively, the reference diagnostic result may be generated using a diagnostic result generation model (e.g., a trained neural network model).

In some embodiments, the first server(s) 110 may obtain the training material from an imaging system (e.g., a single modality imaging system, a multi-modality imaging system) and/or other resources (e.g., a hospital information system (HIS), a picture archiving and communication system (PACS), and a radiology information system (RIS) of a hospital). In some embodiments, the HIS of a hospital may store information relating to subjects treated in the hospital, the PACS of the hospital may include medical images generated in the hospital and information relating to the medical images, and the RIS of the hospital may include reference diagnostic results with respect to medical images.

In some embodiments, a first server 110 may include a data acquisition module for obtaining training material. For example, the data acquisition module may obtain training material from a HIS, a PACS, and a RIS of a hospital in real-time or regularly (e.g., hourly, daily, weekly, monthly) according to an actual situation (e.g., a count of medical images generated in the hospital). Merely by way of example, based on an identification of a medical image of a patient or an identification of the patient, the data acquisition module may acquire the medical image and information relating to the medical image from the PACS, information relating to the patient from the HIS, and a reference diagnostic result with respect to the medical image from the RIS. In some embodiments, the PACS, the RIS, and the HIS of the hospital may be constructed according to a digital imaging and communications in medicine (DICOM) protocol, and data acquired from these systems may not need to be normalized, which may simplify the acquisition and management of training material. Optionally, private information of the patient, such as the name and the ID card of the patient may be anonymized using a natural language processing (NLP) technology.

In some embodiments, the first server(s) 110 may be a single server or a server group. The server group may be centralized or distributed. For example, the first server(s) 110 may include a plurality of distributed servers each of which may be deployed in an information department or a network room of a three-A hospital in China. A first server 110 deployed in a three-A hospital may be configured to collect training material relating to medical images generated in the three-A hospital. As used herein, a three-A hospital refers to a top hospital authenticated by the Ministry of Public Health of China. It should be noted that the term “three-A hospital” is merely used as an example of hospitals having a certain scale (e.g., measured by the count of hospital departments, the count of patients, the count of medical personnel, or the like, or any combination thereof). In some embodiments, the training material corrected by a first server 110 of a hospital may be stored in a local storage device of the hospital and/or the second server 120. In some embodiments, the first server 120 may be implemented by a computing device 200 having one or more components as described in connection with FIG. 2.

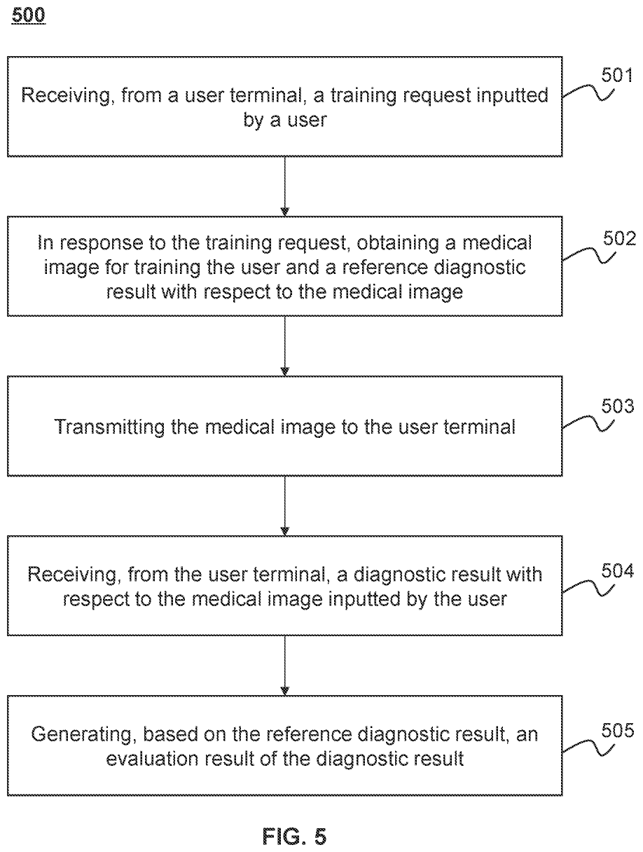

The second server 120 may be configured to process and manage information obtained from the first server(s) 110 and/or the terminal(s) 130. Additionally or alternatively, the second server 120 may be configured to implement methods for medical diagnosis training disclosed herein (e.g., process 500 as described in connection with FIG. 5). For example, the second server 120 may receive a training request from a terminal 130, which is inputted by a user of the terminal 130. In response to the training request, the second server 120 may obtain a medical image for training the user and a reference diagnostic result with respect to the medical image. The second server 120 may transmit the medical image to the terminal 130, and receive a diagnostic result with respect to the medical image inputted by the user from the terminal 130. The second server 120 may further generate an evaluation result of the diagnostic result based on the reference diagnostic result. As another example, the second server 120 may store and manage training material collected by the first server(s) 110. Merely byway of example, the second server 120 may classify the training material collected by the first server(s) 110 into a plurality of groups, e.g., groups corresponding to different imaging modalities, groups corresponding to different imaging parts, or the like, or any combination thereof.

In some embodiments, the second server 120 may be a single server or a server group. The server group may be centralized or distributed. For example, the distributed server group may have a block chain structure, and each node in the block chain structure may perform an exemplary method described in the present disclosure. In some embodiments, the second server 120 may include a cloud server implemented on a cloud platform. The cloud platform may provide a cloud computing server, a cloud database, a cloud storage device, or the like. As an example, the cloud platform may include a private cloud, a public cloud, a hybrid cloud, a community cloud, a distributed cloud, an inter-cloud, a multi-cloud, or the like, or a combination thereof. In some embodiments, the second server 120 may be implemented by a computing device 200 having one or more components as described in connection with FIG. 2.

In some embodiments, the second server 120 may include a data storage/management module, an evaluation module, and a user management module. The storage/management module may store training material obtained from the first server(s) 110, for example, in a relational database. Additionally or alternatively, the storage/management module may also recommend specific training material to a terminal 130 in response to a training request inputted by a user of the terminal 130. The recommendation may be performed based on a preset recommendation rule and/or a user profile (e.g., user preference information as described in 601) of the user. In some embodiments, the recommendation may be performed using a knowledge graph (KG) technique and/or a text recommendation technique.

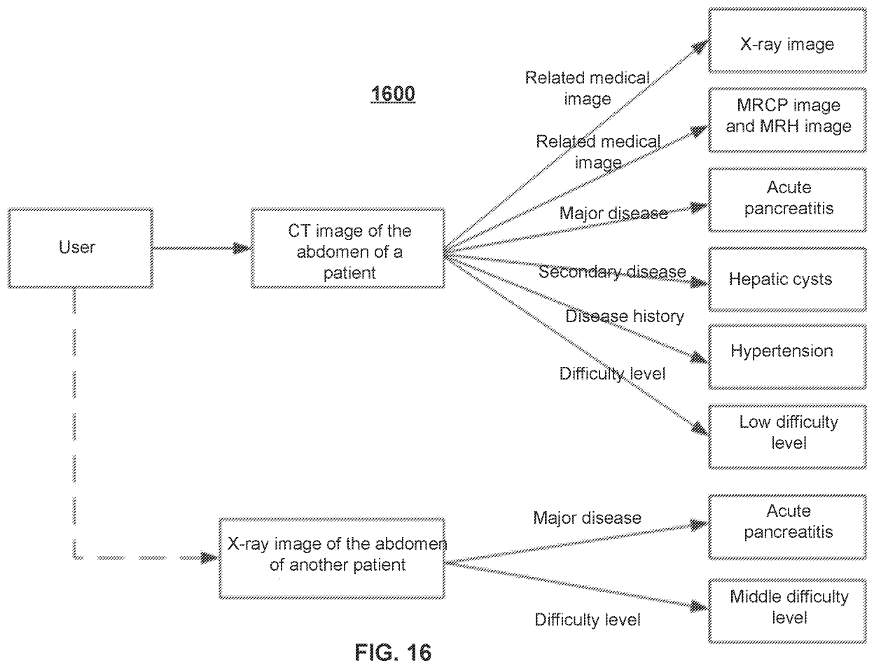

In some embodiments, the storage/management module of the second server 120 may generate a KG (e.g., a KG as shown in FIG. 16) based on the training material. The KG may include a plurality of nodes and a plurality of edges. Each node may represent, for example, a medical image in the training material, information relating to the medical image (e.g., the difficulty level, the disease type, the imaging modality corresponding to the medical image), or information relating to the subject of the medical image. Each of the edges may connect two nodes and represent a relationship (e.g., an inclusion relationship, an attribute relationship) between the two connected nodes. Merely by way of example, a node representing a patient and a node representing a medical image of the patient may be connected by an edge to indicate that the medical image is an image of the patient. As another example, a node representing a medical image may be connected to a node representing the difficulty level of the medical image. As yet another example, two nodes representing two medical images of a same patient may both be connected to a node representing the patient. As yet another example, two nodes representing two medical images corresponding to a same type of disease may both be connected to a node representing the type of disease. As still another example, two nodes representing two medical images having a same difficulty level may both be connected to a node representing the difficulty level. In some embodiments, the second server 120 may update the KG based on newly-obtained training material in real-time or intermittently, such as regularly (e.g., hourly, daily, weekly, monthly).

In some embodiments, the storage/management module of the second server 120 may classify the training material into a plurality of groups according to, for example, the imaging modality, the imaging part, the disease, the difficulty level, the hospital department, or the like, or any combination thereof. Merely by way of example, the training material may be classified into a first group corresponding to a high difficulty level, a second group corresponding to a middle difficulty level, and a third group corresponding to a low difficulty level according to the difficulty level of each medical image in the training material. The difficulty level of a medical image may be obtained from a first server that collects the medical image and/or determined by the second server. For example, the second server 120 may transform the reference diagnostic result of the medical image into a vector or a sequence of vectors using a word embedding technique (e.g., a word2vec tool, a bidirectional encoder representation from transformer (BERT) model). The second server 120 may also extract contextual semantic feature(s) from the vector or the sequence of vectors using, for example, a recurrent neural network (RNN), a convolutional neural network (CNN), along short term memory (LSTM) model, a transformer, or the like. The second server 120 may further determine the difficulty level of the medical image based on the contextual semantic feature(s) using a softmax function. As another example, the second server 120 may obtain a difficulty level determination model. The difficulty level determination model may be a trained model (e.g., a classification model) generated using a machine learning algorithm by the second server 120 or another computing device (e.g., a processing device of a vendor of the difficulty level determination model). The difficulty level determination model may receive the medical image and/or the reference diagnostic result of the medical image as an input, and output the difficulty level of the medical image.

The evaluation component may be configured to generate an evaluation result of a diagnostic result with respect to a medical image based on a reference diagnostic result with respect to the medical image. For example, the evaluation component may generate the evaluation result using an NLP technique and/or a computer vision technique. In some embodiments, the evaluation component may generate the reference diagnostic result by evaluating the medical image using a diagnostic result generation model. The diagnostic result generation model may be generated using an AI technique. For example, the diagnostic result generation model may be generated by training a preliminary model using a machine learning algorithm.

The user management component may be configured to manage users of the training system 100. For example, the user management component may management registration information and/or login information of a user of the training system 100. As another example, the user management component may determine a diagnosis level of the user based on historical training records of the user. If the user reaches a certain diagnosis level, the user management component may issue a level certificate to the user as an incentive. Additionally or alternatively, the user management component may determine an access permission that allows the user to access certain training material. For example, different users may be allowed to access training materials of different hospitals and/or different departments.

The terminal(s) 130 may be configured to enable a user interaction between a user and the training system 100. For example, the terminal(s) 130 may receive a training request inputted by a user. As another example, the terminal(s) 130 may receive the diagnostic result with respect to a medical image inputted by the user, and transmit the diagnostic result to the second server 120 for further processing. As another example, the terminal(s) 130 may receive an evaluation result of the diagnostic result from the second server 120 and display the evaluation result to the user. In some embodiments, the terminal(s) 130 may be connected to and/or communicate with the first server(s) 110 and/or the second server 120. In some embodiments, the terminal(s) 130 may include a mobile device 130-1, a laptop computer 130-2, or the like, or a combination thereof. For example, the mobile device 130-1 may include a mobile phone, a personal digital assistant (PDA), a gaming device, a navigation device, a point of sale (POS) device, a laptop, a tablet computer, a desktop, or the like, or a combination thereof. In some embodiments, the terminal(s) 130 may include an input device, an output device, etc. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to the second server 120 via, for example, a bus, for further processing. Other types of the input device may include a cursor control device, such as a mouse, a trackball, or cursor direction keys, etc. The output device may include a display, a speaker, a printer, or the like, or a combination thereof. In some embodiments, the terminal(s) 130 may belong to a user who needs to receive medical diagnosis training (e.g., a doctor of a township hospital and a countryside hospital) or an organization where the user works.

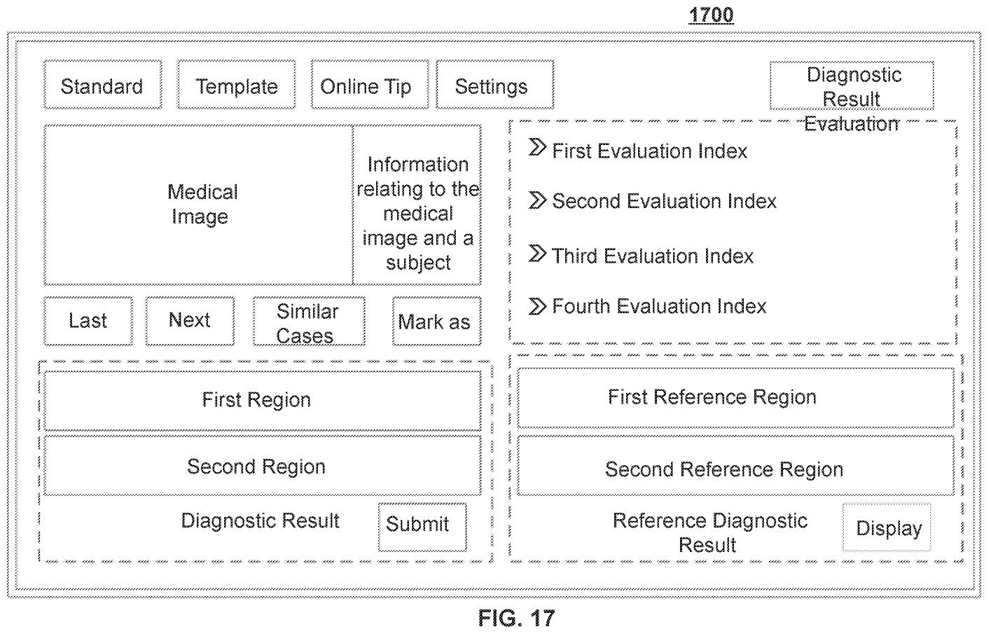

In some embodiments, a terminal 130 may be installed with an operating system (OS), one or more applications (apps), an executable file, or the like. The OS may include a window OS (e.g., a Linux OS, a Mac OS developed by Apple Inc.), a mobile OS (e.g., an iOS™, an Android™), or the like. The executable file may include a medical diagnosis training executable file. The app(s) may include a medical diagnosis training APP, a browser, or the like, or any combination thereof. For example, the medical diagnosis training app may include a registration module, a login module, and a training module. Through the registration module, a user may register a user account by providing a hospital name, a department name, a training direction, a user name, a password, or the like, or any combination thereof. Through the login module, the user may log in the medical diagnosis training APP by inputting the user name, the password, the hospital name, the department name, or the like, or any combination thereof. Optionally, the login module may determine an initial grade regarding the diagnostic capacity of the user by testing the user using a testing medical image. The sequent training of the user (e.g., the selection of training material for the user) may be designed based on the initial grade of the user. Through the training module, the user may input a training request, view a medical image and/or other training material, input a diagnostic result with respect to the medical image, and/or view an evaluation result regarding the diagnostic result. In some embodiments, the training module may include a user interface for displaying information and receiving user input, such as a user interface 1700 as shown in FIG. 17.

The network 140 may include any suitable network that can facilitate the exchange of information and/or data for the training system 100. In some embodiments, one or more components of the training system 100 (e.g., the first server(s) 110, the second server 120, the terminal(s) 130) may communicate information and/or data with one or more other components of the training system 100 via the network 140. For example, the second server 120 may obtain training material from the first server(s) 110 via the network 140. As another example, the second server 120 may obtain a diagnostic result with respect to a medical image inputted by a user from the terminal(s) 130 via the network 140. The network 140 may include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, or the like, or a combination thereof. For example, the network 140 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or a combination thereof. In some embodiments, the network 140 may include one or more network access points. For example, the network 140 may include wired and/or wireless network access points such as base stations and/or internet exchange points through which one or more components of the training system 100 may be connected to the network 140 to exchange data and/or information.

In an exemplary application scenario, the first server(s) 110 may include a plurality of first servers 110 deployed in a plurality of three-A hospitals, and the terminal(s) 130 may include a plurality of terminals 130 deployed in a plurality of township hospitals. The second server 120 may obtain training material from the first servers 110, and transmit the training material (or a portion thereof) to the terminals 130. In some embodiments, the second sever 120 may obtain training material from a certain three-A hospital, and transmit the training material (or a portion thereof) to terminal(s) 130 of one or more township hospitals that have an affiliation with the certain three-A hospital.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. However, those variations and modifications do not depart the scope of the present disclosure. In some embodiments, the training system 100 may include one or more additional components and/or one or more components described above may be omitted. Additionally or alternatively, two or more components of the training system 100 may be integrated into a single component. For example, a component of the training system 100 may be replaced by another component that can implement the functions of the component.

In some embodiments, the training system 100 may include a plurality of second servers 120 each of which is configured to process data obtained from one or more certain first servers 110 and/or one or more certain terminals 130, such as first server(s) 110 and terminal(s) 130 within a certain region. In some embodiments, a terminal 130 may be directly connected to one or more first server(s) 110. For example, a first server 110-1 may have the functions of the second server 120, and be configured to process data (e.g., a training request, a diagnostic result) received from a terminal 130. In some embodiments, the second server 120 may be omitted or integrated into a first server 110.

FIG. 2 is a schematic diagram illustrating exemplary hardware and/or software components of a computing device 200 according to some embodiments of the present disclosure. The computing device 200 may be used to implement any component of the training system 100 as described herein. For example, a first server 110, the second server 120, and/or a terminal 130 may be implemented on the computing device 200, respectively, via its hardware, software program, firmware, or a combination thereof. Although only one such computing device is shown, for convenience, the computer functions relating to the training system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program code) and perform functions of the computing device 200 (e.g., the second server 120) in accordance with techniques described herein. The computer instructions may include, for example, routines, programs, objects, components, data structures, procedures, modules, and functions, which perform particular functions described herein. For example, the processor 210 may process data/information relating to the training system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information relating to the training system 100. In some embodiments, the storage device 220 may be connected to the network 140 to communicate with one or more other components in the training system 100 (e.g., the first server(s) 110, the second server 120, and/or the terminal(s) 130). One or more components of the training system 100 may access the data or instructions stored in the storage device 220 via the network 140. In some embodiments, the storage device 220 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), a digital versatile disk ROM, etc. In some embodiments, the storage device may be implemented on a cloud platform as described elsewhere in the disclosure. In some embodiments, the storage device 220 may store one or more programs and/or instructions to perform exemplary methods described in the present disclosure. For example, the storage device 220 may store a program for the computing device 200 to execute for medical diagnosis training.

The I/O 230 may input and/or output signals, data, information, etc. In some embodiments, the I/O 230 may enable a user interaction with the second server 120. In some embodiments, the I/O 230 may include an input device and an output device. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to another component (e.g., the second server 120) via, for example, a bus, for further processing. Other types of the input device may include a cursor control device, such as a mouse, a trackball, or cursor direction keys, etc. The output device may include a display (e.g., a liquid crystal display (LCD), a light-emitting diode (LED)-based display, a flat panel display, a curved screen, a television device, a cathode ray tube (CRT), a touch screen), a speaker, a printer, or the like, or a combination thereof.

The communication port 240 may be connected to a network (e.g., the network 140) to facilitate data communications. The communication port 240 may establish connections between the computing device 200 and one or more other components of the training system 100 (e.g., a first server 110, a terminal 130). The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee™ link, a mobile network link (e.g., 3G, 4G, 5G), or the like, or a combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

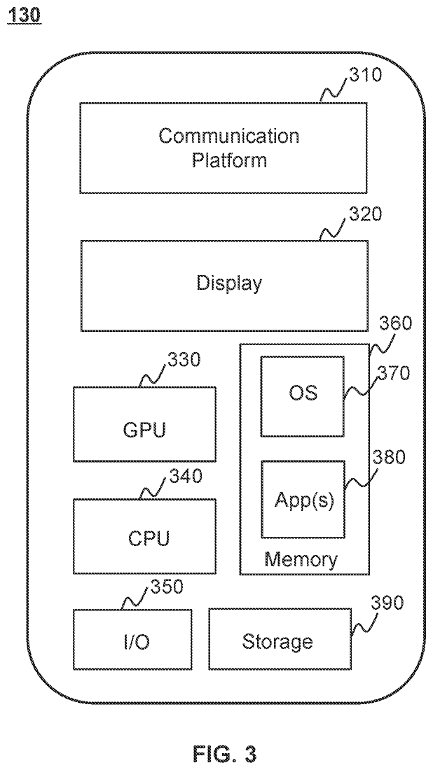

FIG. 3 is a schematic diagram illustrating exemplary hardware and/or software components of a mobile device 300 according to some embodiments of the present disclosure. In some embodiments, one or more components (e.g., a terminal 130 and/or the second server 120) of the training system 100 may be implemented on the mobile device 300.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to the training system 100. User interactions with the information stream may be achieved via the I/O 350 and provided to the second server 120 and/or other components of the training system 100 via the network 140.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

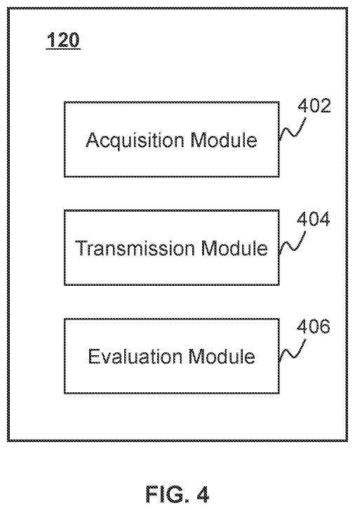

FIG. 4 is a block diagram illustrating an exemplary second server 120 according to some embodiments of the present disclosure. As shown in FIG. 4, the second server 120 may include an acquisition module 402, a transmission module 404, and an evaluation module 406.

The acquisition module 402 may be configured to receive or obtain information relating to the training system 100. For example, the acquisition module 402 may receive a training request inputted by a user from a user terminal (e.g., a terminal 130). The acquisition module 402 may be also configured to obtain a medical image for training the user and a reference diagnostic result with respect to the medical image. The acquisition module 402 may be further configured to receive a diagnostic result with respect to the medical image inputted by the user from the user terminal. More descriptions regarding the acquisition of the training request, the medical image, the reference diagnostic result, and the diagnostic result may be found elsewhere in the preset disclosure. See, e.g., operations 501, 502, and 504 and relevant descriptions thereof.

The transmission module 404 may be configured to transmit the medical image to the user terminal. For example, the transmission module 404 may transmit the medical image to the user terminal via a network (e.g., the network 140).

The evaluation module 406 may be configured to generate an evaluation result of the diagnostic result based on the reference diagnostic result. The evaluation may be performed to evaluate, for example, the accuracy, the format, the integrity, the clarity, the logic, or the like, or any combination thereof, of the diagnostic result. Merely by way of example, the evaluation result of the diagnostic result may include a value of a first evaluation index relating to a description order of the diagnostic result, a value of a second evaluation index relating to a content integrity of the diagnostic result, a value of a third evaluation index relating to an accuracy of the diagnostic result, or a value of a fourth evaluation index relating to a similarity between the diagnostic result and the reference diagnostic result, or the like, or any combination thereof. In some embodiments, the evaluation module 406 may determine the value of each of the four evaluation indexes, and determine the evaluation result by performing a weighted algorithm on the values of the four evaluation indexes. More descriptions regarding the generation of the evaluation result of the diagnostic result may be found elsewhere in the preset disclosure. See, e.g., 505 and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the second server 120 may include one or more additional modules, such as a storage module (not shown) for storing data. As another example, one or more modules of the second server 120 described above may be omitted. Additionally or alternatively, two or more modules of the second server 120 may be integrated into a single component. A module of the second server 120 may be divided into two or more units.

In some embodiments, the second server 120 may further include a training module configured to train one or more trained models (e.g., a difficulty level determination model, a diagnostic result generation model) used in medical training. For example, the training module may use one or more machine learning algorithms to generate the trained model(s). Exemplary machine learning algorithms may include an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, a rule-based machine learning algorithm, or the like, or any combination thereof. The machine learning algorithm used to generate the gradient waveform determination model may be a supervised learning algorithm, a semi-supervised learning algorithm, an unsupervised learning algorithm, or the like. In some embodiments, the training module may be implemented by another processing device, e.g., a processing device a vendor who provides and/or maintains such a trained model.

FIG. 5 is a flowchart illustrating an exemplary process 500 for medical diagnosis training according to some embodiments of the present disclosure. In some embodiments, the process 500 may be executed by the training system 100. For example, the process 500 may be implemented as a set of instructions (e.g., an application) stored in a storage device (e.g., the storage device of the second server 120, the storage device 220, and/or the storage 390). In some embodiments, the second server 120 (e.g., a processing device of the second server 120, the processor 210 of the computing device 200, and/or one or more modules illustrated in FIG. 4) may execute the set of instructions and may accordingly be directed to perform the process 500.

In 501, the second server 120 (e.g., the acquisition module 402) may receive, from a user terminal (e.g., a terminal 130), a training request inputted by a user.

The training request may direct the second server 120 to initiate a medical diagnosis training process for the user. The training request may include, for example, a user ID of the user, a terminal ID of the terminal, preference information of the user (e.g., a type of medical image that the user wants to view), or the like, or any combination thereof. In some embodiments, the user may input the training request into the user terminal by performing a triggering operation (e.g., pressing a specific button on the user terminal, inputting an instruction into a box displayed on an interface of the user terminal, inputting a voice instruction via the user terminal).

In 502, in response to the training request, the second server 120 (e.g., the acquisition module 402) may obtain a medical image for training the user and a reference diagnostic result with respect to the medical image.

The medical image for training the user may be an image of a subject (e.g., a patient) acquired by an imaging modality as described elsewhere in this disclosure, such as CT, MRI, PET, PET-CT, PET-MRI, X-ray, ultrasound, or the like. The medical image may correspond to a specific department of a hospital, such as a neurology department, a neurosurgery department, a hepatobiliary department, a pancreatic surgery department, a urogenital department, a gastroenterology department, or the like. The medical image may be an image of an imaging part, such as a specific organ, a specific tissue, or the like, of the subject. Exemplary imaging parts may include the brain, the neck, the chest, the abdomen, the pelvis, the lower limb, or the like, of the subject. The medical image may be an image of a subject that has a specific disease, such as cerebral hemorrhage, coronary stenosis, a prostate cancer, an acute pancreatitis, a pulmonary nodule, or the like, or any combination thereof. The medical image may have a specific difficulty level (e.g., a high difficulty level, a middle difficulty level, a low difficulty level).

The reference diagnostic result with respect to the medical image may have a desired quality (e.g., a desired accuracy, a standard format), which may be used as a reference substance in the evaluation of a diagnostic result with respect to the medical image inputted by the user. For example, the reference diagnostic result may be provided or confirmed by a senior doctor (e.g., a doctor having a work experience more than certain years, a doctor having a senior title, a doctor who has passed a test of medical diagnosis, etc.). As another example, the reference diagnostic result may be generated by inputting the medical image into a diagnostic result generation model.

In some embodiments, the second server 120 may also obtain information relating to the medical image (e.g., the difficulty level, the hospital department corresponding to the medical image), information (e.g., the name, a medical record, an imaging part) relating to the subject corresponding to the medical image, or the like, or any combination thereof. More descriptions regarding the information relating to a medical image and/or a subject corresponding to the medical image may be found elsewhere in the present disclosure. See, e.g., FIG. 1 and relevant descriptions thereof.

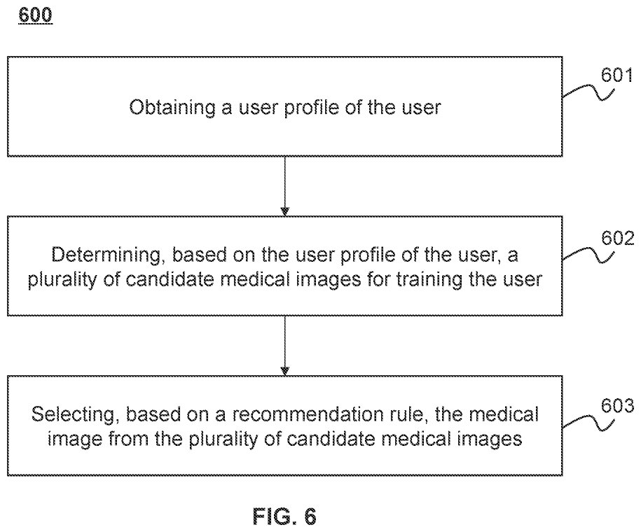

In some embodiments, the medical image, the reference diagnostic result, and the information relating to the medical image may be obtained from a storage device of the second server 120, an imaging system of a hospital, a storage device of a first server 110, or the like, or any combination thereof. In some embodiments, the second server 120 may select the medical image from a plurality of candidate images randomly or according to a specific rule. For example, the second server 120 may obtain the medical image based on a user profile of the user and a recommendation rule, which will be described in detail in FIG. 6. In some embodiments, the second server 120 may obtain a plurality of medical images, and each of the plurality of medical images may be used to train the user by performing operations 503-505 described below.

In 503, the second server 120 (e.g., the transmission module 404) may transmit the medical image to the user terminal.

In some embodiments, the second server 120 may transmit the medical image to the user terminal via a network (e.g., the network 140).

In 504, the second server 120 (e.g., the acquisition module 402) may receive, from the user terminal, a diagnostic result with respect to the medical image inputted by the user.

For example, after the medical image is received, the user terminal may display the medical image and optionally the information relating to the medical image on a user interface (e.g., a user interface 1700 shown in FIG. 17). The user may view the medical image and the information relating to the medical image, and input the diagnostic result via the user terminal. The user terminal may then transmit the diagnostic result to the second server 120 through a network (e.g., the network 140). In some embodiments, the diagnostic result may include a first part that describes the content of the medical image and/or a second part that describes a diagnostic opinion regarding the subject. For example, the first part may include descriptions regarding an anatomical structure in the medical image, a feature of the anatomical structure, a feature value of the feature, or the like, or any combination thereof. The second part may include a diagnostic conclusion. The diagnostic conclusion may be a concluding statement regarding a detected lesion, for example, a disease, an abnormal state of the imaging part of the subject.

In 505, the second server 120 (e.g., the evaluation module 406) may generate an evaluation result of the diagnostic result based on the reference diagnostic result.

The evaluation may be performed to evaluate, for example, the accuracy, the format, the integrity, the clarity, the logic, or the like, or any combination thereof, of the diagnostic result. Merely by way of example, the evaluation result of the diagnostic result may include value(s) of one or more evaluation indexes, such as a value of a first evaluation index relating to a description order of the diagnostic result, a value of a second evaluation index relating to a content integrity of the diagnostic result, a value of a third evaluation index relating to an accuracy of the diagnostic result, or a value of a fourth evaluation index relating to a similarity between the diagnostic result and the reference diagnostic result, or the like, or any combination thereof. The value of a specific evaluation index may be represented as a score, a level, or the like, or any combination thereof.

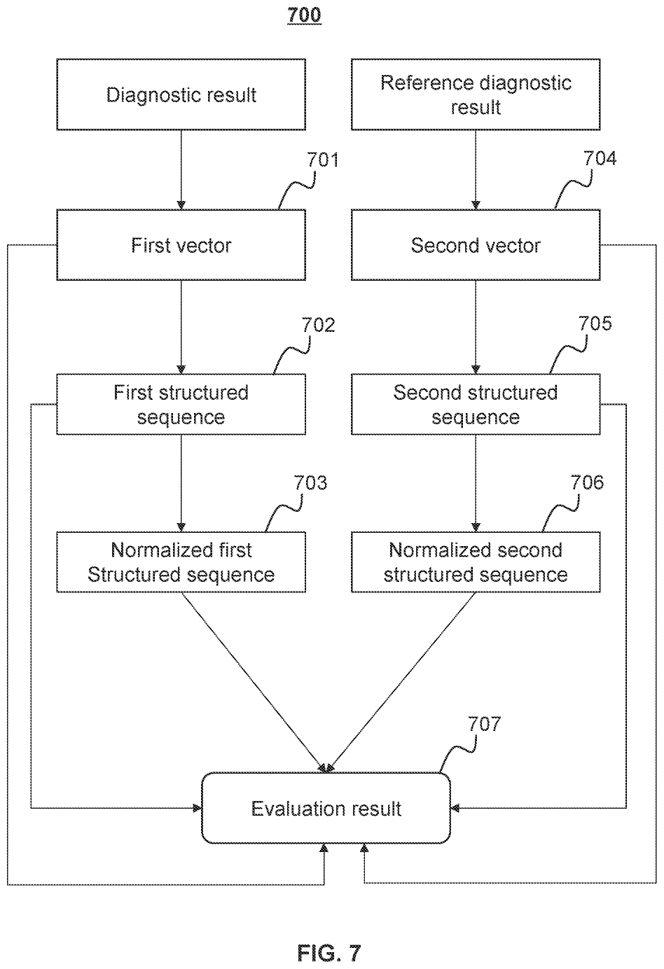

In some embodiments, the evaluation index(es) included in the evaluation result may be selected according to the default setting of the training system 100, automatically selected by the second server 120, or selected by the user or a person who is responsible for training the user. In some embodiments, the second server 120 may determine the value of each of a plurality of evaluation indexes, and determine the evaluation result by performing a weighted algorithm (e.g., a weighted average, a weighted sum) on the values of the evaluation indexes. In the weighted algorithm, the weight of an evaluation index may reflect the influence degree of the evaluation index on the quality of the diagnostic result. In some embodiments, the second server 120 may generate the evaluation result using an NLP technique and/or a computer version technique. More descriptions regarding the generation of the evaluation result may be found elsewhere in the present disclosure. See, e.g., FIGS. 7-11 and relevant descriptions thereof.

It should be noted that the above description regarding the process 500 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 500 may be accomplished with one or more additional operations not described and/or without one or more of the operations discussed above. For example, before operation 501, the second server 120 may cause the user terminal to display a prompt for reminding the user to complete a medical diagnosis training.

FIG. 6 is a schematic diagram illustrating an exemplary process 600 for obtaining a medical image for training a user according to some embodiments of the present disclosure. In some embodiments, the process 600 may be executed by the training system 100. For example, the process 600 may be implemented as a set of instructions (e.g., an application) stored in a storage device (e.g., the storage device of the second server 120, the storage device 220, and/or the storage 390). In some embodiments, the second server 120 (e.g., the processing device of the second server 120, the processor 210 of the computing device 200, and/or one or more modules illustrated in FIG. 4) may execute the set of instructions and may accordingly be directed to perform the process 600. In some embodiments, one or more operations of the process 600 may be performed to achieve at least part of operation 502 as described in connection with FIG. 5.

In 601, the second server 120 (e.g., the acquisition module 402) may obtain a user profile of the user.

The user profile may include personal data of the user, for example, basic information, user preference information, information relating to one or more historical medical images that have been used to train the user, one or more historical diagnostic results inputted by the user, a historical evaluation result regarding each historical diagnostic result inputted by the user, a diagnosis level of the user, or the like, or any combination thereof. The basic information may include, for example, e.g., the name, the gender, the age, a department that the user belongs to, a working experience, a job title, or the like, or any combination thereof. The user preference information may include, for example, a difficulty level, an imaging modality, a disease, an imaging part (e.g., the abdomen, the chest), a patient group (e.g., patients under the age of 10), a senior doctor, a hospital, or the like, or any combination thereof, that the user is interested in. The information relating to one or more historical medical images that have been used to train the user may include an imaging device or an imaging modality that generates each historical medical image, an imaging condition under which each historical medical image is generated, information relating to a subject associated with each historical medical image (e.g., an imaging part, a disease, or the like, of the subject), a difficulty level of each historical medical image, a hospital department corresponding to each historical medical image, distribution information of the types of the historical medical image(s), distribution information of the difficulty levels of the historical medical image(s), or the like, or any combination thereof.

In some embodiments, the user profile (or a portion thereof) may be manually set by the user. For example, the user may input his/her preference information into the user terminal. Additionally or alternatively, the user profile (or a portion thereof) may be generated by a computing device (e.g., the second server 120) based on, for example, a medical diagnostic test and/or a questionnaire conducted by the user, historical training records of the user, or the like, or any combination thereof. In some embodiments, the user profile (or a portion thereof) may be stored in a storage (e.g., a storage device of the second server 120, the storage device 220, and/or the storage 390) and be retrieved by the second server 120 for further processing.

In 602, the second server 120 (e.g., the acquisition module 402) may determine a plurality of candidate medical images for training the user based on the user profile of the user.

In some embodiments, as described in connection with FIG. 1, the second server 120 may store training material obtained from the first server(s) 110. The second server 120 may select the candidate medical images from training material randomly or according to a specific selection rule. For example, the selected candidate medical images may fit the preference of the user. Merely by way of example, according to the user profile, the user is interested in CT imaging modality, the brain, and a middle difficulty level. The selected candidate medical images may include one or more CT images of the human brain with a middle difficulty level.

In some embodiments, the training material stored in the second server 120 may be classified into a plurality of groups according to, for example, the imaging modality, the imaging part, the disease, the difficulty level, the hospital department, or the like, or any combination thereof. The candidate medical images may be selected from the training material according to the classification of the training material and the user profile. For example, if the user is interested in CT imaging modality, the candidate medical images may be selected from a group of training material corresponding to CT imaging modality.

In some embodiments, the second server 120 may determine the candidate medical images using a KG technique. For example, a knowledge graph may be generated based on the training material stored in the second server 120, and the candidate medical images may be determined based on the knowledge graph using a semantic path-based ranking algorithm for recommendation (Sprank). Merely by way of example, the user profile of the user may be mapped on the knowledge graph to identify nodes and/or edges that fit the preference of the user, and the medical images corresponding to the identified nodes and/or edges may be designated as the candidate medical images. More descriptions regarding the knowledge graph may be found elsewhere in the present disclosure. See, e.g., FIG. 1 and relevant descriptions thereof. In some embodiments, the training material and/or the knowledge graph may be stored in a specific device other than the second server 120 (e.g., a storage device of a first server 110 or an external storage device), and the second server 120 may retrieve the training material and/or the knowledge graph from the specific device.

In 603, the second server 120 (e.g., the acquisition module 402) may select the medical image from the plurality of candidate medical images based on a recommendation rule.

The recommendation rule may be associated with a recommended imaging modality, a recommended disease type, a recommended imaging part, and a recommended ratio, a recommended difficulty level, or the like, or any combination thereof. For example, the recommendation rule may specify that medical images recommended to the user are CT images. As another example, the recommendation rule may specify that medical images recommended to the user need to be generated by various imaging modalities. As yet another example, the recommendation rule may specify that medical images corresponding to one or more complications of a specific disease need to be recommended to the user if the user is interested in the specific disease. As yet another example, the recommendation rule may specify a preset time (e.g., 10 o'clock every night) for recommending the medical image. As used herein, a recommended ratio refers to a ratio of a count of medical images that have not been used to train the user to a count of historical medical images that have been mistakenly diagnosed by the user. For example, if the recommendation rule may specify that the recommendation ratio is 3/1, one historical medical image that has been mistakenly diagnosed by the user may be recommended to the user after 3 medical images that have not been used to train the user are recommended to the user.

In some embodiments, the recommendation rule may be determined according to a defaulting setting of the training system 100, or set by the user manually, or determined by the second server 120 according to an actual need. For example, the recommendation rule may be determined by the second server 120 according to the user profile of the user. In some embodiments, the user profile of the user may be updated continuously or intermittently (e.g., periodically), and the recommendation rule may be adjusted with the update of the user profile. For example, if the diagnosis level of the user is improved, the recommended difficulty level and/or the recommended ratio in the recommendation rule may be adjusted accordingly. In some embodiments, the recommendation rule may be adjusted automatically by the second server 120 or manually by a mentor of the user. More descriptions regarding the selection of the medical image from the plurality of candidate medical images may be found elsewhere in the present disclosure. See, e.g., FIG. 16 and relevant descriptions thereof.

In some embodiments, by using a specific recommendation rule, medical images recommended to the user may have a suitable distribution in, for example, the imaging modality, the difficulty level, the disease, or the like, or any combination thereof. For example, medical images captured by various imaging modalities and/or corresponding to various diseases may be used to train the user, thereby improving the diversity of the medical images and the training effect. As another example, medical images having a specific difficulty level matching the diagnosis level of the subject may be used to train the user. In this way, the user may be trained using more targeted medical images, which may improve the training effect. In addition, the recommendation rule may be determined and/or updated according to the user profile of the user, thereby achieving customized and effective training.

It should be noted that the above description regarding the process 600 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 600 may be accomplished with one or more additional operations not described and/or without one or more of the operations discussed above. For example, operation 603 may be omitted. In operation 602, the second server 120 may directly select the medical image for training the user from the training material according to the user profile of the user and/or the recommendation rule. In some embodiments, the second server 120 may update the user profile of the user and/or the recommendation rule in real-time or intermittently (e.g., regularly).

FIG. 7 is a schematic diagram illustrating an exemplary process for generating an evaluation result of a diagnostic result based on a reference diagnostic result according to some embodiments of the present disclosure. In some embodiments, one or more operations of process 700 as shown in FIG. 7 may be performed to achieve at least part of operation 505 as described in connection with FIG. 5.

As shown in FIG. 7, the diagnostic result and the reference diagnostic result may be processed, respectively, to generate various data representations (e.g., a vector, a structured sequence, a normalized structured sequence). The evaluation result may be generated based on the data representations of the diagnostic result and the reference diagnostic result.

The processing of the diagnostic result may include operations 701, 702, and 703. In 701, the second server 120 may generate a first vector representing the diagnostic result. For example, the first vector may be a numerical matrix generated using a word embedding technique. Merely by way of example, the second server 120 may segment the diagnostic result into a plurality of words using a tokenization technique. The second server 120 may further generate a word vector for each of the plurality of words using the word embedding technique. The first vector may be generated by combining the word vectors corresponding to the plurality of words.

In 702, the second server 120 may determine one or more first structured sequences based on the first vector. Each of the first structured sequence(s) may include a first anatomical structure, a first feature of the first anatomical structure, and a first feature value of the first feature recorded in the diagnostic result.

The first anatomical structure of a first structured sequence may include a medical entity, such as an imaging part of the subject (e.g., an organ, a tissue). Exemplary first anatomical structures may include the head, the neck, a blood vessel, a liver, or the like, of the subject. Exemplary first features of a first anatomical structure may include a density shadow, a shape, a boundary, a signal intensity, a position, a size, a thickness, a wall, a surface, an expansion, or the like, or any combination thereof. A first feature value of a first feature may include a numerical value, a determination result, a classification, or the like, or any combination thereof, relating to the first feature. For example, the first feature value of the size may include an area value or a volume value. As another example, the first feature value of the shape may include a determination result as to whether the shape is uniform, thickening, expanding, or irregular. As yet another example, the first feature value of the density shadow may be a determination result as to whether there is a density shadow. As still another example, the first feature value of the boundary may include a determination as to whether the boundary is clear.

Merely byway of example, the diagnostic result may include a first statement “at the transverse section of the left middle abdomen at the level of the 4th lumbar vertebra, a slightly higher density shadow having a small nodule shape can be seen.” In the first statement, the “transverse section of the left middle abdomen at the level of the 4th lumbar vertebra” may represent a first anatomical structure, the “density shadow” may represent a feature of the first anatomical structure, the “slightly higher” may represent the feature value of the signal intensity of the “density shadow”, and the “small nodule shape” may represent the feature value of the shape of the “density shadow.”

As another example, the diagnostic result may include a second statement “the thorax is symmetrical, the transparency of the lung fields is good, no abnormal density shadow is observed in the lung fields, and the hilar shadows are not large.” In the second statement, “the thorax” and “the lung fields” may represent first anatomical structures, the “transparency,” the “abnormal density shadow,” and “the hilar shadows” may represent features of the “the lung fields,” “symmetrical” may represent the feature value of the position of the thorax, “good” may represent the feature value of the transparency of the lung fields, “no” may represent the feature value of the abnormal density shadow, and “not large” may represent the feature value of the size of the hilar shadows. As another example, the diagnostic result may include a third statement “the heart shadow has a normal shape, both sides of the diaphragm are smooth, and the costophrenic angle is sharp.” In the third statement, the “heart” and “both sides of the diaphragm” may represent first anatomical structures, the “shape” and the “costophrenic angle” may represent features of the heart, “smooth” may represent the feature value of the smoothness of the both sides of the diaphragm, and “sharp” may represent the feature value of “the costophrenic angle”.

In some embodiments, the second server 120 may recognize a plurality of terms from the diagnostic result, and generate the first structured sequence(s) by combining the terms. For example, a first structured sequence may be represented as “a first anatomical structure-a first feature of the first anatomical structure-a first feature value of the first feature.” Merely by way of example, the diagnostic result may include a fourth statement “the intracranial and cervical vascular walls are smooth, there is no obvious sign of stenosis or dilation, and there is no obvious abnormity in the main branches.” Two low-enhanced regions are observed at the right thyroid gland, one of the enhanced regions has a size of 8 millimeters (mm)*8 mm, and the other one of the enhanced regions has a size of 4 mm*2 mm.” The second server 120 may recognize terms including “intracranial,” “cervical,” “vascular,” “stenosis,” “dilation,” “no,” etc., from the diagnostic result. The second server 120 may further combine the terms into a plurality of first structured sequences including “the intracranial and cervical vascular-walls-smooth,” “the right thyroid gland-the size of one of the low-enhanced regions-8 mm*8 mm,” “right thyroid gland-the size of the other one of the low-enhanced regions-4 mm*2 mm,” and “the intracranial and cervical vascular-dilation-no.”

In some embodiments, the diagnostic result may include a plurality of paragraphs that are described in a first order in the diagnostic result. Each paragraph may include one or more sentences that are described in a second order in the diagnostic result. The second server 120 combines the terms of the diagnostic result into a plurality of first structured sequences, and further rank the first structured sequences according to the first order and the second order. In some embodiments, the plurality of first structured sequences may be combined into an integrated first structured sequence, which includes the first structured sequences ranked according to the first order and the second order.

In some embodiments, the diagnostic result may include a statement describing a first feature value of a first anatomical structure without directly mentioning the first feature corresponding to the first feature value. Merely by way of example, the diagnostic result may include a statement “the thyroid gland is large,” in which the “thyroid gland” is a first anatomical structure and the “large” is a feature value of the size of the “thyroid gland.” The second server 120 may determine the first feature corresponding to the first feature value, and generate a first structured sequence based on the determined first feature. Alternatively, the second server 120 may generate a first structured sequence in which the first feature is a null value.

In some embodiments, the second server 120 may determine the first structured sequence(s) using a semantic labeling technique. For example, the first structured sequence(s) may be determined by inputting the first vector into a trained sequence labeling model (e.g., an RNN model, an LSTM model).

In 703, for each of the first structured sequence(s), the second server 120 may generate a normalized first structured sequence by normalizing the first structured sequence.

In some embodiments, for a first structured sequence, the second server 120 may generate the corresponding normalized first structured sequence by normalizing the first anatomical structure and the first feature in the first structured sequence using a standard dictionary. The standard dictionary may include standard terms used in medical diagnosis. For example, for each of a plurality of anatomical structures of human, the standard dictionary may include a standard term representing the anatomical structure, synonyms of the standard term representing the anatomical structure, a standard term representing a feature of the anatomical structure, and synonyms of the standard term representing the feature of the anatomical structure dimension. For example, exemplary standard terms relating to the brain may include the cerebral hemisphere, the basal ganglia, the brainstem, the cerebellum, the midline shift, the cistern, etc. As another example, exemplary standard terms relating to a feature of the brain may include the density, the boundary, the shape, the size, the thickness, etc.

Based on the standard dictionary, the second server 120 may determine a standard term corresponding to the first anatomical structure (also referred to as a normalized first anatomical structure) and a standard term corresponding to the first feature (also referred to as a normalized first feature). The second server 120 may generate the normalized first structured sequence by combining the normalized first anatomical structure, the normalized first feature, and the first feature value. In this way, the terms in the diagnostic result may be normalized, thereby eliminating or reduce the effect of polysemy in the evaluation of the diagnostic result. In some embodiments, if there is no standard term corresponding to the first anatomical structure and/or the first feature in the standard dictionary, the second server 120 may generate the normalized first structured sequence using the original first anatomical structure and/or the original first feature. Optionally, the second server 120 or another computing device may update the standard dictionary by adding a standard term corresponding to the first anatomical structure and/or a standard term corresponding to the first feature.

In some embodiments, the standard dictionary may be previously generated by the second server 120 or another computing device (e.g., the computing device 200) based on, for example, expert consensus, a writing standard in medical diagnosis. The standard dictionary may be stored in a storage device (e.g., the storage device of the second server 120, the storage device 220, and/or the storage 390), and retrieved by the second server 120. Optionally, the standard dictionary may be updated continuously or intermittently (e.g., periodically).

In some embodiments, a plurality of standard dictionaries each of which corresponds to a certain imaging part and/or a certain imaging modality may be previously generated. The second server 120 may select a standard dictionary according to information relating to the medical image for training the user, and normalize the first structured sequence(s) using the selected standard dictionary. For example, if the medical image is a CT image, the second server 120 may select a standard dictionary corresponding to the CT imaging modality. As another example, if the medical image is an image of the head of a patient, the second server 120 may select a standard dictionary corresponding to the head of human.

The processing of the reference diagnostic result may include operations 704, 705, and 706 as shown in FIG. 7.

In 704, the second server 120 may generate a second vector representing the reference diagnostic result. Operation 704 may be performed in a similar manner as operation 701, and the descriptions thereof are not repeated here.

In 705, the second server 120 may determine one or more second structured sequences based on the second vector. Each of the second structured sequence(s) may include a second anatomical structure, a second feature of the second anatomical structure, and a second feature value of the second feature recorded in the reference diagnostic result. Operation 705 may be performed in a similar manner as operation 702, and the descriptions thereof are not repeated here.

In 706, for each of the second structured sequence(s), the second server 120 may generate a normalized second structured sequence by normalizing the second structured sequence. For example, for a second structured sequence, the second server 120 may normalize the second anatomical structure and the second feature in the second structured sequence, so as to generate a normalized second structured sequence including the normalized second anatomical structure, the normalized second feature, and the original second feature value of the second structured sequence. Operation 706 may be performed in a similar manner as operation 703, and the descriptions thereof are not repeated here. In some embodiments, if the reference diagnostic result is generated using the diagnostic result generation model, the second structured sequence(s) of the reference diagnostic result may be directly determined as the normalized second structured sequence(s).

In 707, the second server 120 may generate the evaluation result of the diagnostic result.

The evaluation result of the diagnostic result may be generated based on one or more of the first vector, the second vector, the first structured sequence(s), the second structured sequence(s), the normalized first structured sequence(s), and the normalized second structured sequence(s). For example, as described in connection with FIG. 5, the evaluation result may include value(s) of one or more evaluation indexes, such as a first evaluation index relating to a description order of the diagnostic result, a second evaluation index relating to a content integrity of the diagnostic result, a third evaluation index relating to an accuracy of the diagnostic result, and/or a fourth evaluation index relating to a similarity between the diagnostic result and the reference diagnostic result. The values of different evaluation indexes may be determined based on a same set of data or different sets of data.

Merely by way of example, the second server 120 may determine the value of the fourth evaluation index relating to the similarity between the diagnostic result and the reference diagnostic result by determining a similarity between the first vector and the second vector. The similarity between the first vector and the second vector may be measured by, for example, a vector distance (e.g., a Euclidean distance, a cosine distance) between the first vector and the second vector. As another example, the second server 120 may determine the value of the second evaluation index relating to a content integrity of the diagnostic result based on the first structured sequence(s) and the second structured sequence(s). As yet another example, the value(s) of one or more evaluation indexes may be determined based on the normalized first structured sequence(s) and the normalized second structured sequence(s). It should be noted that the examples described herein are merely provided for illustration purposes, and not intended to be limiting. The evaluation of the diagnostic result performed based on specific data representations of the diagnostic result and the reference diagnostic result may be also performed based on other data representations of the diagnostic result and the reference diagnostic result.

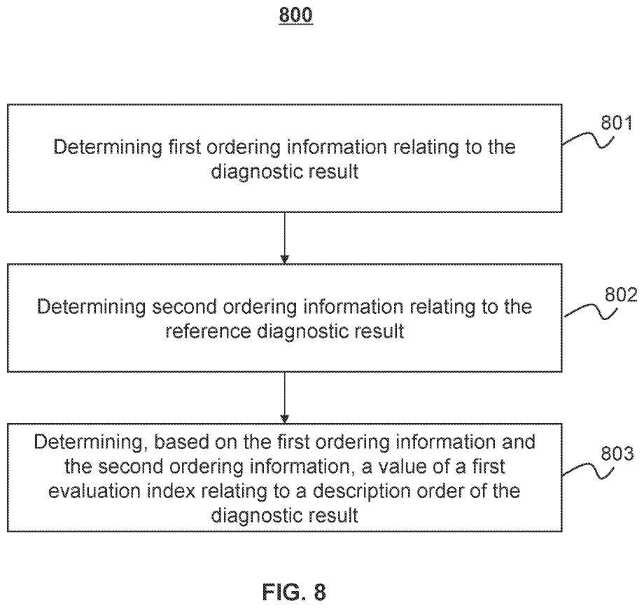

FIG. 8 is a flowchart illustrating an exemplary process 800 for determining a value of a first evaluation index relating to a description order of a diagnostic result according to some embodiments of the present disclosure. In some embodiments, the process 800 may be executed by the training system 100. For example, the process 800 may be implemented as a set of instructions (e.g., an application) stored in a storage device (e.g., the storage device of the second server 120, the storage device 220, and/or the storage 390). In some embodiments, the second server 120 (e.g., the processing device of the second server 120, the processor 210 of the computing device 200, and/or one or more modules illustrated in FIG. 4) may execute the set of instructions and may accordingly be directed to perform the process 800. In some embodiments, one or more operations of the process 800 may be performed to achieve at least part of operation 505 as described in connection with FIG. 5.

For illustration purposes, the determination of the value of the first evaluation index based on the first structured sequence(s) and the second structured sequence(s) is described hereinafter as an example. It should be noted that the value of the first evaluation index may be also determined based on other data representations of the diagnostic result and the reference diagnostic result, such as the normalized first structured sequence(s) and the normalized second structured sequence(s).

The first evaluation index may be used to evaluate whether the description order of the diagnostic result is consistent with a recommended (or standard) description order. As used herein, the description order of a diagnostic result may include a description order of a plurality of first anatomical structures in the diagnostic result, a description order of a plurality of first features of a first anatomical structure in the diagnostic result, or the like, or any combination thereof. Generally, different anatomical structures may have different importance and/or pathological changes, and it is desired that these anatomical structures are described in a recommended (or standard) description order in the diagnostic result. For example, an anatomical structure having an abnormal sign and/or being a key organ need to be described firstly in the diagnostic result. Merely by way of example, in a diagnostic result of an abdominal image, the anatomical structures of the abdomen may be described in the order of the liver, the lobes of the liver, the intrahepatic bile ducts, the gallbladder, the pancreas, the spleen, and the double kidneys. Additionally or alternatively, different features of an anatomical structure may have different importance, and it is desired that these features are described in a recommended (or standard) description order.

In some embodiments, the recommended description order may be set manually, determined according to the default setting of the training system 100, or determined by the second server 120 according to an actual need. For example, because the reference diagnostic result is provided or confirmed by a senior doctor, or generated by a diagnostic result generation model, the description order of the reference diagnostic result (e.g., second ordering information as described in connection with 802) may be determined and regarded as the recommended description order. For illustration purposes, the following descriptions describe the determination of the value of the first evaluation index based on the reference diagnostic result as an example. This is not intended to be limiting. In some embodiments, the value of the first evaluation index may be determined based on a recommended description order which is set by, for example, a senior doctor, a medical organization, or the like.

In 801, the second server 120 (e.g., the evaluation module 406) may determine first ordering information relating to the diagnostic result.

The first ordering information may reflect an order of appearance of the first anatomical structure(s) of the first structured sequence(s) in the diagnostic result and/or an order of appearance of the first feature(s) of the first anatomical structure(s) in the diagnostic result. For example, the first ordering information may indicate that in the diagnostic result, the left lung, the right lung, and the liver are described in sequence, and the size, the shape, and the position of the left lung are described in sequence. In some embodiments, the second server 120 may determine the first ordering information based on the first structured sequence(s) determined in operation 702.

In 802, the second server 120 (e.g., the evaluation module 406) may determine second ordering information relating to the reference diagnostic result.

The second ordering information may reflect an order of appearance of the second anatomical structure(s) of the second structured sequence(s) in the reference diagnostic result and/or an order of appearance of the second feature(s) of the second anatomical structure(s) in the reference diagnostic result. In some embodiments, the second server 120 may determine the second ordering information based on the second structured sequence(s) determined in operation 705.

In 803, the second server 120 (e.g., the evaluation module 406) may determine the value of the first evaluation index relating to the description order of the diagnostic result based on the first ordering information and the second ordering information.

In some embodiments, the second server 120 may determine the value of the first evaluation index relating to the description order by comparing the first ordering information and the second ordering information. For example, the second server 120 may determine a first difference between the order of appearance of the first anatomical structure(s) in the diagnostic result and the order of appearance of the second anatomical structure(s) in the reference diagnostic result. Additionally or alternatively, the second server 120 may determine one or more anatomical structures that are described in both the diagnostic result and the reference diagnostic result, which may be referred to as common anatomical structure(s) for the convenience of descriptions. For each common anatomical structure, the second server 120 may determine a second difference between the order of appearance of the first feature(s) of the common anatomical structure in the diagnostic result and the order of appearance of the second feature(s) of the common anatomical structure in the reference diagnostic result. The second server 120 may then determine the value of the first evaluation index relating to the description order based on the first difference and/or the second difference(s) of the common anatomical structure(s). In some embodiments, the second server 120 may generate a first string corresponding to the diagnostic result based on the first order information and a second string corresponding to the reference diagnostic result based on the second order information. For example, the first string may include the first anatomical structure(s) that are arranged according to their order of appearance in the diagnostic result, and the second string may include the second anatomical structure(s) that are arranged according to their order of appearance in the reference diagnostic result. As another example, the first string may include first feature(s) of a common anatomical structure that are arranged according to their order of appearance in the diagnostic result, and the second string may include second feature(s) that are arranged according to their order of appearance in the reference diagnostic result. The second server 120 may determine a difference between the first string and the second string, and determine the value of the first evaluation index based on the edit distance. For example, the difference may be measured by an edit distance between the first string and the second string. The edit distance refers to a count of operations required to transform the first string to the second string. A higher edit distance may indicate a higher difference between the description order of the diagnostic result and the description order of the reference diagnostic result.

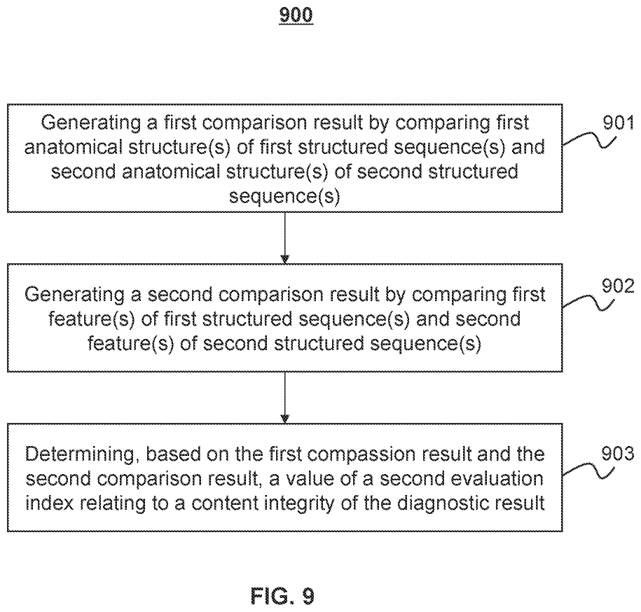

FIG. 9 is a flowchart illustrating an exemplary process 900 for determining a value of a second evaluation index relating to a content integrity of a diagnostic result according to some embodiments of the present disclosure. In some embodiments, the process 900 may be executed by the training system 100. For example, the process 900 may be implemented as a set of instructions (e.g., an application) stored in a storage device (e.g., the storage device of the second server 120, the storage device 220, and/or the storage 390). In some embodiments, the second server 120 (e.g., the processing device of the second server 120, the processor 210 of the computing device 200, and/or one or more modules illustrated in FIG. 4) may execute the set of instructions and may accordingly be directed to perform the process 900. In some embodiments, one or more operations of the process 900 may be performed to achieve at least part of operation 505 as described in connection with FIG. 5.

For illustration purposes, the determination of the value of the second evaluation index based on the first structured sequence(s) and the second structured sequence(s) is described hereinafter as an example. It should be noted that the value of the second evaluation index may be also determined based on other data representations of the diagnostic result and the reference diagnostic result, such as the normalized first structured sequence(s) and the normalized second structured sequence(s).

The second evaluation index may be used to evaluate the content integrity of the diagnostic result, for example, whether the diagnostic result includes all the second anatomical structure(s) and/or all the second feature(s) mentioned in the reference diagnostic result.

In 901, the second server 120 (e.g., the evaluation module 406) may generate a first comparison result by comparing the first anatomical structure(s) of the first structured sequence(s) and the second anatomical structure(s) of the second structured sequence(s).

In some embodiments, the second server 120 may determine a third difference between the count of the first anatomical structure(s) and the count of the second anatomical structure(s). For example, if there are 4 first anatomical structures and 5 second anatomical structures, the third difference may be 1, which may reflect an incompleteness of the diagnostic result. Additionally or alternatively, for each of the second anatomical structure(s), the second server 120 may determine a fourth difference by determining whether there is a first anatomical structure corresponding to the second anatomical structure. As used herein, if a first anatomical structure corresponds to a same medical entity (e.g., organ or tissue) as a second anatomical structure, the first anatomical structure may be deemed as corresponding to the second anatomical structure, and the first anatomical structure and the second anatomical structure may be regarded as a common anatomical structure described in both the diagnostic result and the reference diagnostic result. If there is a first anatomical structure corresponding to a specific second anatomical structure, the fourth difference corresponding to the specific second anatomical structure may be determined to be 0; and if there is no first anatomical structure corresponding to the specific second anatomical structure, the fourth difference may be determined to be 1. The second server 120 may determine the first comparison result based on the third difference and the fourth difference(s). For example, the second server 120 may determine a first sum (e.g., a weighted sum) of the third difference and the fourth difference(s). A higher first sum may indicate that more content is missing in the diagnostic result.

In 902, the second server 120 (e.g., the evaluation module 406) may generate a second comparison result by comparing the first feature(s) of the first structured sequence(s) and the second feature(s) of the second structured sequence(s).

In some embodiments, for each common anatomical structure both mentioned in the diagnostic result and the reference diagnostic result, the second server 120 may generate a second comparison result by comparing the first feature(s) and the second feature(s) of the common anatomical structure. For example, for a specific common anatomical structure, the second server 120 may determine a fifth difference between the count of the first feature(s) and the count of the second feature(s). For example, both the diagnostic result and the reference diagnostic result mention the lungs of a patient. If the diagnostic result describes 2 first features of the lungs and the reference diagnostic result describes 3 second features of the lungs, the fifth difference corresponding to the lungs may be 1, which may reflect an incompleteness of the diagnostic result. Additionally or alternatively, for each second feature of the specific common anatomical structure, the second server 120 may determine a sixth difference by determining whether there is a first feature corresponding to the second feature. As used herein, if a first feature and a second feature are the same or synonyms, the first feature may be deemed as corresponding to the second feature, and the first feature and the second feature may be regarded as a common feature described in both the diagnostic result and the reference diagnostic result. If there is a first feature corresponding to a second feature, the sixth difference of the second feature may be determined to be 0; and if there is no first feature corresponding to the second feature, the sixth difference of the second feature may be determined to be 1. The second server 120 may determine the second comparison result based on the fifth difference and the sixth difference(s) of each common anatomical structure. For example, the second server 120 may determine a second sum (e.g., a weighted sum) of the fifth difference and the sixth difference(s) of each common anatomical structure.

In 903, the second server 120 (e.g., the evaluation module 406) may determine a value of the second evaluation index relating to the content integrity of the diagnostic result based on the first compassion result and the second comparison result.

For example, the second server 120 may determine the value of the second evaluation index by performing a weighted algorithm on the first comparison result (e.g., the first sum) and the second comparison result (e.g., the second sum). In some embodiments, operation 902 may be omitted, and the value of the second evaluation index may be determined merely based on the first comparison result. Alternatively, operation 901 may be omitted, and the value of the second evaluation index may be determined merely based on the second comparison result.

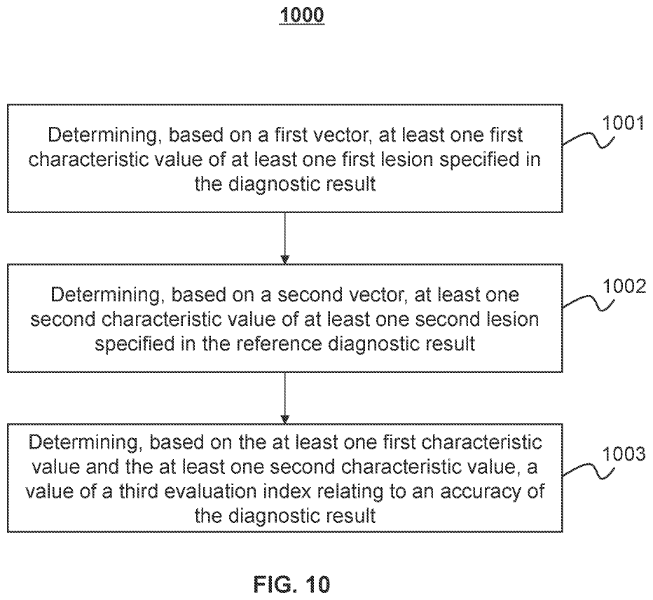

FIG. 10 is a flowchart illustrating an exemplary process 1000 for determining a value of a third evaluation index relating to an accuracy of a diagnostic result according to some embodiments of the present disclosure. In some embodiments, the process 1000 may be executed by the training system 100. For example, the process 1000 may be implemented as a set of instructions (e.g., an application) stored in a storage device (e.g., the storage device of the second server 120, the storage device 220, and/or the storage 390). In some embodiments, the second server 120 (e.g., the processing device of the second server 120, the processor 210 of the computing device 200, and/or one or more modules illustrated in FIG. 4) may execute the set of instructions and may accordingly be directed to perform the process 1000. In some embodiments, one or more operations of the process 1000 may be performed to achieve at least part of operation 505 as described in connection with FIG. 5.

In 1001, the second server 120 (e.g., the evaluation module 406) may determine at least one first characteristic value of at least one first lesion specified in the diagnostic result based on the first vector.

The at least one first characteristic value of the at least one first lesion may include one or more first characteristic values of each first lesion. A lesion refers to a portion of a subject that has a damage (or potential damage) and/or an abnormal change (or potential change), usually caused by disease or trauma. Exemplary lesions may include a soft-tissue lesion, a diabetes-associated lesion, a bone lesion, a brain lesion, a skin lesion, a gastrointestinal lesion, an endodermal lesion, or the like, or any combination thereof. A characteristic value of a lesion may include a positioning value, a quantitative value, a qualitative value, or the like, or any combination thereof. The positioning value may describe the position of the lesion, for example, a left upper lobe, a left lower lobe. The qualitative value may be descriptive, for example, be represented in the form of a non-real-valued expression (e.g., a character, a string) that describes the lesion. For example, exemplary qualitative values may include whether the lesion is malignant or benign, whether the lesion is calcified or not calcified, or the like. The quantitative value may be obtained using a quantifiable measurement process, for example, be represented in the form of a real-valued expression (e.g., a numerical value, a mathematical formula, a mathematical model, a grade). For example, exemplary quantitative values may include that the size of the lesion is 5 mm*4 mm. Merely by way of example, a diagnostic result of a coronary CT angiography image may include a fifth statement “a calcified plaque is present at the proximal end of a left anterior descending (LAD) artery, and is moderately narrow.” In the fifth statement, the “proximal end of a left anterior descending (LAD) artery” may be a positioning value, the “calcified plaque” may be a qualitative value, and the “moderately narrow” may be a quantitative value.

In some embodiments, the second server 120 may determine the first characteristic value(s) of a first lesion based on a first structured sequence. The first structured sequence may be generated based on the first vector as described in connection with operation 702. For example, the first anatomical structure may include a first anatomical structure, a first feature of the first anatomical structure, and/or a first feature value of the first feature. The elements of the first structured sequence may indicate a lesion of a subject and/or characteristics of the lesion, and can be used to determine the first characteristic value(s). Taking the fifth statement aforementioned as an example, “the proximal end of a left anterior descending (LAD) artery” may represent a first anatomical structure, the “calcified plaque” may represent a first feature of “the proximal end of a left anterior descending (LAD) artery”, and the “moderately narrow” may represent a first feature value of the “calcified plaque”. The second server 120 may determine that the lesion is located at the proximal end of the LAD artery (i.e., the positioning value) based on the first anatomical structure, and the lesion has a calcified plaque (i.e., a qualitative value) that is moderately narrow (i.e., a quantitate value) based on the first feature and the first feature value. In some embodiments, the second server 120 may determine the first characteristic value(s) of a first lesion based on a normalized first structured sequence.

Additionally or alternatively, the diagnostic result may include a diagnostic conclusion that describes a detected disease or lesion, an abnormal state of an imaging part of a subject, etc. The second server 120 may determine the first characteristic value(s) of a first lesion based on the diagnostic conclusion. For example, a diagnostic result regarding a prostate may include a diagnostic conclusion “the patient possibly has a prostate cancer and a prostate hyperplasia.” Based on the diagnostic conclusion, the second server 120 may determine that qualitative values of the prostate include the “prostate cancer” and the “prostate hyperplasia.”

In 1002, the second server 120 (e.g., the evaluation module 406) may determine at least one second characteristic value of at least one second lesion specified the reference diagnostic result based on the second vector.

The at least one second characteristic value of the at least one second lesion may include one or more second characteristic values of each second lesion. In some embodiments, the second server 120 may determine the second characteristic value(s) of a second lesion based on a (normalized) second structured sequence and/or a diagnostic conclusion in the reference diagnostic result. The (normalized) second structured sequence may be generated based on the second vector as described in connection with FIG. 7. Additionally or alternatively, the second server 120 may determine the second characteristic value(s) of the second lesion using an AI technique. For example, the second characteristic value(s) may be determined by inputting the medical image into a lesion detection model (e.g., a CNN model). The lesion detection model may be a trained model or an algorithm configured to receive an image as an input, and identify a lesion and/or determine feature information (e.g., the position, the size) of the lesion based on the image. In some embodiments, the at least one second characteristic value may be determined based on both the reference diagnostic result and the AI technique, so as to improve the accuracy of the value of the third evaluation index determined based thereon.

In 1003, the second server 120 (e.g., the evaluation module 406) may determine a value of the third evaluation index relating to the accuracy of the diagnostic result based on the at least one first characteristic value and the at least one second characteristic value.

In some embodiments, for each second lesion described in the reference diagnostic result, the second server 120 may determine whether there is a first lesion corresponding to the second lesion. If a first lesion and a second lesion correspond to a same medical entity (e.g., organ or tissue), the first lesion may be regarded as corresponding to the second lesion, and the first lesion and the second lesion may be regarded as a common lesion described in both the diagnostic result and the reference diagnostic result. For example, the second server 120 may determine a first count of second lesions that are described in the diagnostic result (i.e., have corresponding first lesions), and determine a true positive rate by determining a ratio of the first count to the total count of the second lesion(s). The second server 120 may determine a higher value of the third evaluation index if the true positive rate is high. As another example, the second server 120 may determine a second count of first lesions that are not described in the reference diagnostic result (i.e., don't have corresponding second lesions), and determine a false positive rate of the diagnostic result by determining a ratio of the second count to the total count of the second lesion(s). The second server 120 may determine a lower value of the third evaluation index if the false positive rate is high.

Additionally or alternatively, for each common lesion described in both the diagnostic result and the reference diagnostic result, the second server 120 may compare the first characteristic value(s) of the common lesion and the second characteristic value(s) of the common lesion. For example, for a specific common lesion, the second server 120 may compare a first characteristic value of the common lesion with a second characteristic value corresponding to the first characteristic value. A first characteristic value and a second characteristic value may be regarded as corresponding to each other if they relate to a same or similar feature of the common lesion. The second server 120 may determine a higher value of the third evaluation index if a first characteristic value is the same as or similar to its corresponding second characteristic value.

In some embodiments, the second server 120 may determine the value of the third evaluation index relating to the accuracy of the diagnostic result by performing a weighted algorithm on, for example, the false positive rate, the true positive rate, the difference between a first characteristic value and its corresponding second characteristic value, or the like, or any combination thereof.

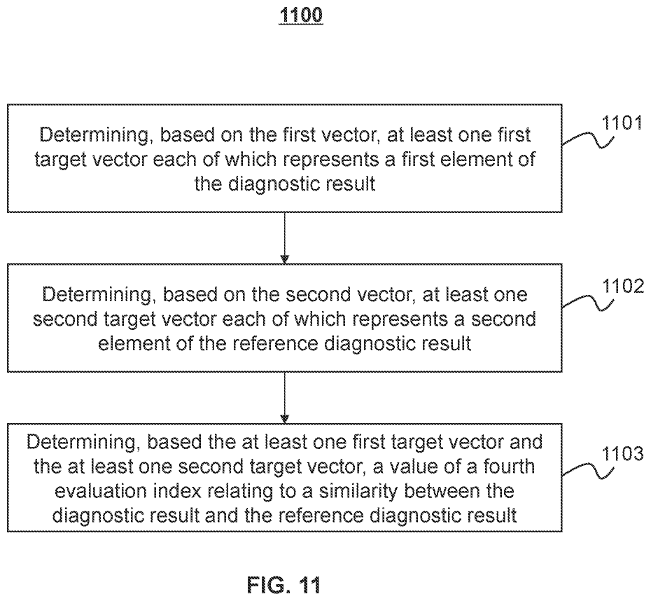

FIG. 11 is a flowchart illustrating an exemplary process 1100 for determining a value of a fourth evaluation index relating to a similarity between a diagnostic result and a reference diagnostic result according to some embodiments of the present disclosure. In some embodiments, the process 1100 may be executed by the training system 100. For example, the process 1100 may be implemented as a set of instructions (e.g., an application) stored in a storage device (e.g., the storage device of the second server 120, the storage device 220, and/or the storage 390). In some embodiments, the second server 120 (e.g., the processing device of the second server 120, the processor 210 of the computing device 200, and/or one or more modules illustrated in FIG. 4) may execute the set of instructions and may accordingly be directed to perform the process 1100. In some embodiments, one or more operations of the process 1100 may be performed to achieve at least part of operation 505 as described in connection with FIG. 5.

In 1101, the second server 120 (e.g., the evaluation module 406) may determine at least one first target vector based on the first vector. Each of the at least one first target vector may represent a first element of the diagnostic result.

Exemplary first elements may include a word, a sentence element, a paragraph, or the like, or any combination thereof, recorded in the diagnostic result. In some embodiments, the second server 120 may determine the at least one first target vector based on the first vector. For example, the second server 120 may determine the first target vector by inputting the first vector into a trained neural network model (e.g., an LSTM model) for generating a target vector. In some embodiments, a plurality of first target vectors representing a plurality of first elements of the diagnostic result may be determined. The first target vectors may be ranked according to the order of appearance of the first elements in the diagnostic result. In some embodiments, the at least one first target vector may be generated based on the diagnostic result instead of the first vector according to, for example, a text analysis technique.

In 1102, the second server 120 (e.g., the evaluation module 406) may determine at least one second target vector based on the second vector. Each of the at least one second target vector may represent a second element of the reference diagnostic result.

The second element may include a word, a sentence, a paragraph, or the like, or any combination thereof, recorded in the reference diagnostic result. In some embodiments, the second server 120 may determine the at least one second target vector based on the second vector. For example, the second server 120 may determine the second target vector by inputting the second vector into the trained neural network model for generating a target vector. In some embodiments, a plurality of second target vectors representing a plurality of second elements of the reference diagnostic result may be determined. The second target vectors may be ranked according to the order of appearance of the second elements in the reference diagnostic result. In some embodiments, the at least one second target vector may be generated based on the reference diagnostic result instead of the second vector according to, for example, a text analysis technique.

In 1103, the second server 120 (e.g., the evaluation module 406) may determine a value of the fourth evaluation index relating to the similarity between the diagnostic result and the reference diagnostic result based on the at least one first target vector and the at least one second target vector.

In some embodiments, for each second element described in the reference diagnostic result, the second server 120 may determine whether there is a first element corresponding to the second element described in the diagnostic result. For example, if two words are the same or synonyms, they may be regarded as corresponding to each other. As another example, if two sentences relate to a same medical entity or have a same order of appearance in the diagnostic result and the reference diagnostic result, they may be regarded as corresponding to each other. For each second element, the second server 120 may further determine a similarity between the second target vector of the second element and the first target vector of the corresponding first element. A similarity between two vectors may be measured by a vector distance (e.g., a Euclidean distance, a cosine distance) there between. A smaller distance may indicate a higher similarity between the two vectors. If there is no first element corresponding to a specific second element, the second server 120 may determine that the similarity corresponding the specific second element is 0. The second server 120 may then determine the value of the fourth evaluation result based on the similarity (or similarities) corresponding to the second element(s), for example, by performing a weighted algorithm on the similarity (or similarities). In some embodiments, the second server 120 may determine the value of the fourth evaluation index by determining a similarity between the first vector and the second vector.

It should be noted that the above descriptions regarding the processes 800 to 1100 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more of the processes 800 to 1100 may be accomplished with one or more additional operations not described and/or without one or more of the operations discussed above.

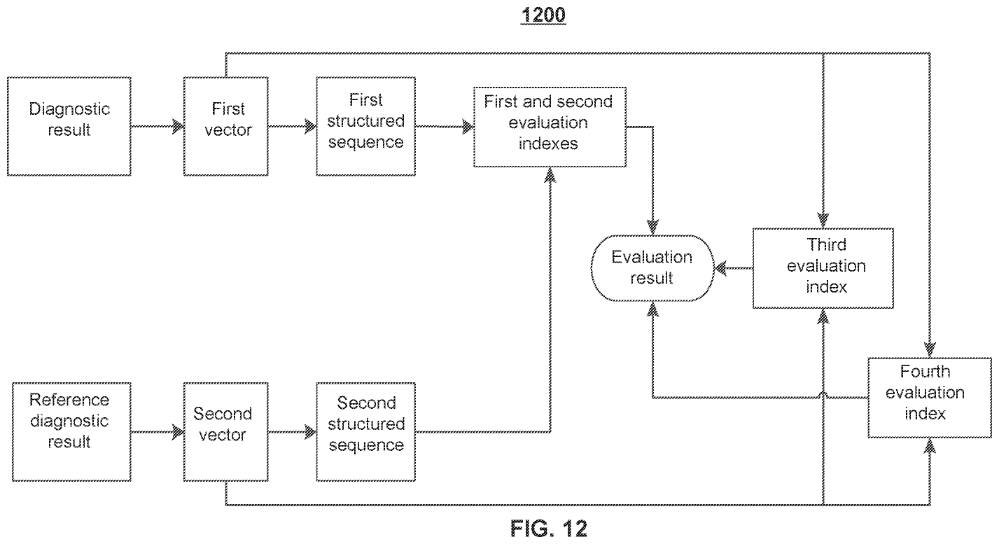

FIG. 12 is a schematic diagram illustrating an exemplary process 1200 for generating an evaluation result of a diagnostic result based on a reference diagnostic result according to some embodiments of the present disclosure. As shown in FIG. 12, the second server 120 may generate a first vector representing the diagnostic result and a second vector representing the reference diagnostic result. The second server 120 may generate one or more first structured sequences based on the first vector, and generate one or more second structured sequences based on the second vector. The second server 120 may determine a value of a first evaluation index relating to a description order of the diagnostic result and a value of a second evaluation index relating to a content integrity of the diagnostic result based on the first structured sequence(s) and the second structured sequence(s). The second server 120 may also determine a value of a third evaluation index relating to an accuracy of the diagnostic result based on the first vector and the second vector. The second server 120 may further determine a value of a fourth evaluation index relating to a similarity between the diagnostic result and the reference diagnostic result based on the first vector and the second vector. The evaluation result may be determined based on the values of the first evaluation index, the second evaluation index, the third evaluation index, and the fourth evaluation index.

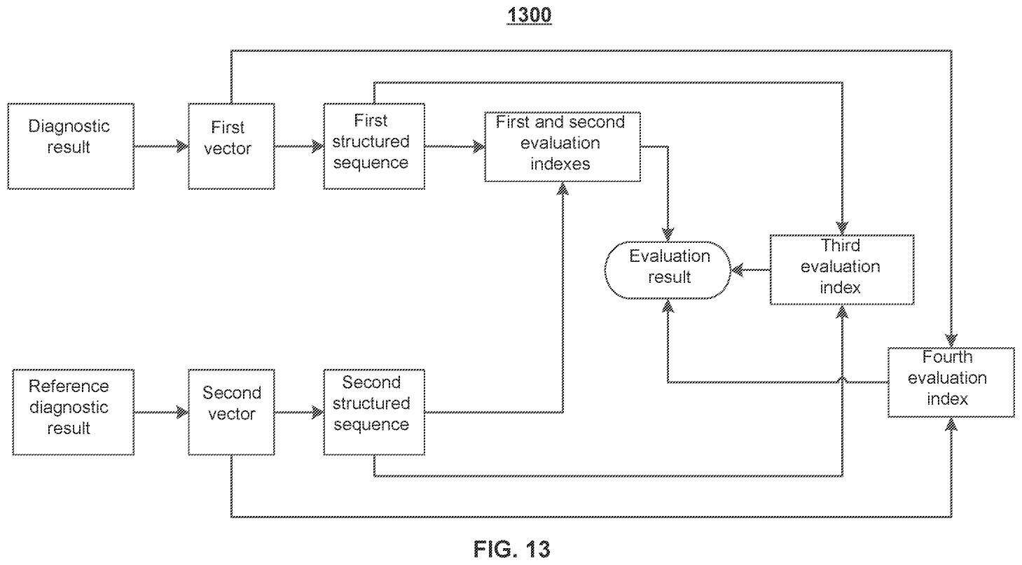

FIG. 13 is a schematic diagram illustrating an exemplary process 1300 for generating an evaluation result of a diagnostic result based on the reference diagnostic result according to some embodiments of the present disclosure. As shown in FIG. 13, the process 1300 may be similar to the process 1200, except that the value of the third evaluation index is determined based on the first structured sequence(s) and the second structured sequence(s).

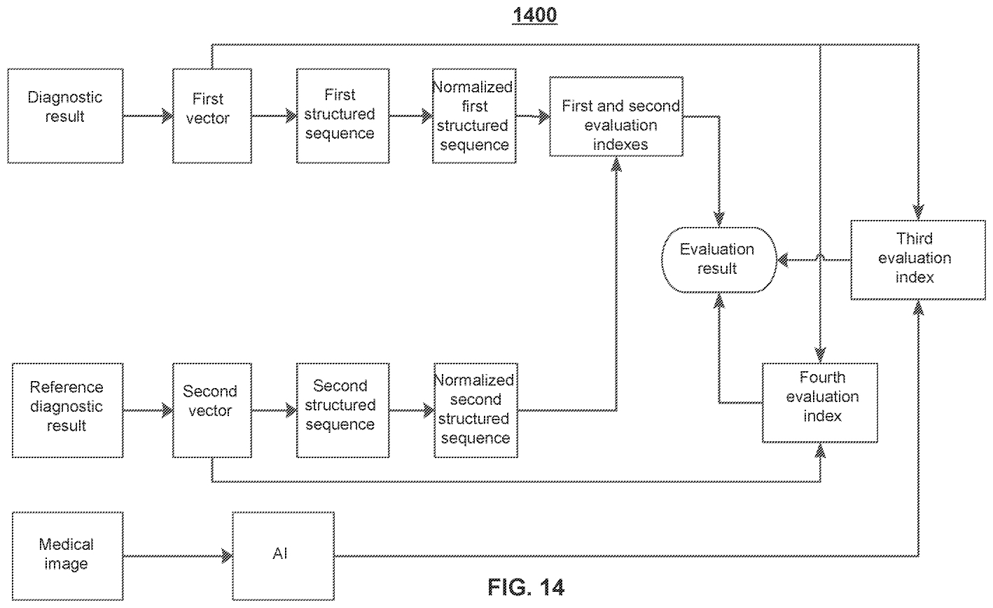

FIG. 14 is a schematic diagram illustrating an exemplary process 1400 for generating an evaluation result of a diagnostic result based on the reference diagnostic result according to some embodiments of the present disclosure. The process 1400 may be similar to the process 1200, except for certain features. As shown in FIG. 14, the second server 120 may normalize the first structured sequence(s) to generate normalized first structured sequence(s), and normalize the second structured sequence(s) to generate normalized second structured sequence(s). The values of the first evaluation index and the second evaluation index may be determined based on the normalized first structured sequence(s) and the normalized second structured sequence(s). The second server 120 may further use an AI technique to analyze the medical image, and determine the value of the third evaluation index relating to the accuracy of the diagnostic result based on the first vector and the analysis result generated by the AI technique. More descriptions regarding the determination of the third evaluation index based on an AI technique may be found elsewhere in the present disclosure. See, e.g., FIG. 10 and relevant descriptions thereof.

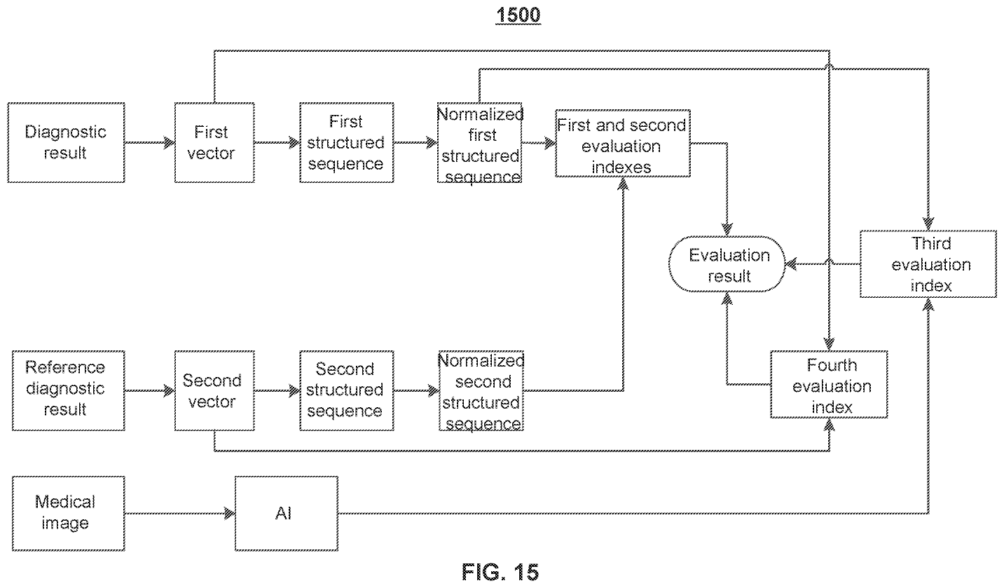

FIG. 15 is a schematic diagram illustrating an exemplary process 1500 for generating an evaluation result of a diagnostic result based on a reference diagnostic result according to some embodiments of the present disclosure. As shown in FIG. 15, the process 1500 may be similar to the process 1400, except that the value of the third evaluation index is determined based on the normalized first structured sequence(s) and the analysis result generated by the AI technique.

FIG. 16 is a schematic diagram illustrating an exemplary process 1600 for selecting a medical image for training a user according to some embodiments of the present disclosure. The process 1600 may be performed to achieve at least part of operation 502 as described in connection with FIG. 5. For illustration purposes, it is assumed that the user profile of the user indicates that the user is interested in an acute abdomen. Symptoms of the acute abdomen may include an intestinal obstruction, a perforation, a calculus, bleeding, and inflammation, etc. Medical images, such as a planar film X-ray image, a CT image (e.g., a planar CT image, an enhanced CT image), and an MRI image, are often used for analyzing the acute abdomen. The second server 120 may determine a medical image for training the user based on the user profile and historical training records of the user.

For example, as shown in FIG. 16, the user has been trained by a CT image of a patient having hypertension. The major disease of the patient is acute pancreatitis, and the secondary disease of the patient is hepatic cysts. Other medical images of the patient include an X-ray image, an MR cholangiopancreatography (MRCP) image, and an MR hydrography (MRH) image. The CT image has a low difficulty level. Based on the historical training records and the user profile, the second server 120 may select an X-ray image of an abdomen of another patient who also has acute pancreatitis, wherein the X-ray image may have a middle difficulty level. In this way, the user may be trained with various types of medical images and an improved difficulty level.

FIG. 17 is a schematic diagram illustrating an exemplary user interface 1700 of a user terminal (e.g., the terminal 130) according to some embodiments of the present disclosure. As shown in FIG. 17, the user interface 1700 may include a region for displaying a medical image of a subject, a region for the user to input a diagnostic result with respect to the medical image, a region for displaying a reference diagnostic result, a region for displaying an evaluation result, a region for displaying various tools (e.g., an online tip button, a submit button), or the like, or any combination thereof.

In some embodiments, before the user submits the diagnostic result with respect to the medical image, the region for displaying the reference diagnostic result may be in an inactive state, for example, be hidden. In some embodiments, the user terminal may further include a display controlling module configured to control the user interface 1700. The display controlling module may cause the user terminal to activate the region for displaying the reference diagnostic result if the second server 120 receives the diagnostic result inputted by the user. Additionally or alternatively, the display controlling module may cause the user terminal to display the evaluation result when the evaluation result is received from the second server 120.

In a training process, the region for displaying the medical image may display the medical image of a subject, information relating to the medical image, and information relating to the subject. The region for displaying the diagnostic result may include a first region for the user to input descriptions regarding the content of the diagnostic result and a second region for the user to input a diagnostic conclusion of the diagnostic result. When the user presses a submit button, the diagnostic result may be uploaded to the second server 120. The second server 120 may generate an evaluation result of the diagnostic result and transmit the evaluation result back to the user terminal. The region for displaying the evaluation result may display the evaluation result (e.g., values of first, second, third, and fourth evaluation indexes). After the diagnostic result is submitted, the user terminal may display the reference diagnostic result on the region for displaying the reference diagnostic result. The region for displaying the reference diagnostic result may include a first reference region for displaying descriptions regarding the content of the reference diagnostic result and a second reference region for displaying a diagnostic conclusion of the reference diagnostic result. In some embodiments, the reference diagnostic result region of the user interface 1700 may include a display button, and the user may need to press the display button to view the reference diagnostic result.

The region for displaying the tools may include a “Standard” button for obtaining a writing standard of the diagnostic result, a “Template” button for obtaining templates of the diagnostic result, an “Online Tip” button for obtaining some tips for writing the diagnostic result, a “Settings” button, a “Last” button for obtaining a last medical image, a “Next” button for obtaining a next medical image, a “Similar Cases” button for obtaining a medical image similar to the current medical image, and a “Mark as” button for marking the current medical image as a typical case or a misdiagnosed case. The “Settings” button may be used to perform a regular setting, a correction setting, or the like. For example, through the regular setting, the user preference information of the user may be inputted into the user terminal, which may be used to construct a user profile. As another example, through the correction setting, a grammatical error (e.g., a spelling error, a punctuation error) may be automatically corrected. Optionally, the region for displaying the diagnostic result may display the grammatical error via a wave underline.

It should be noted that the user interface illustrated in FIG. 17 and the descriptions thereof are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the user interface 1700 may include one or more additional regions, and/or one or more regions described above may be omitted.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Per, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A system for medical diagnosis training, comprising:
at least one storage device including a set of instructions; and
at least one processor configured to communicate with the at least one storage device, wherein when executing the set of instructions, the at least one processor is configured to direct the system to perform operations including:
receiving, from a user terminal, a training request inputted by a user;
in response to the training request, obtaining a medical image for training the user and a reference diagnostic result with respect to the medical image;
transmitting the medical image to the user terminal;
receiving, from the user terminal, a diagnostic result with respect to the medical image inputted by the user; and
generating, based on the reference diagnostic result, an evaluation result of the diagnostic result.

2. The system of claim 1, wherein the evaluation result of the diagnostic result comprises at least one of:
a value of a first evaluation index relating to a description order of the diagnostic result;
a value of a second evaluation index relating to a content integrity of the diagnostic result;
a value of a third evaluation index relating to an accuracy of the diagnostic result, or
a value of a fourth evaluation index relating to a similarity between the diagnostic result and the reference diagnostic result.

3. The system of claim 1, wherein the generating, based on the reference diagnostic result, an evaluation result of the diagnostic result comprises:
generating a first vector representing the diagnostic result;
generating a second vector representing the reference diagnostic result; and
generating, based on the first vector and the second vector, the evaluation result of the diagnostic result.

4. The system of claim 3, wherein the generating, based on the first vector and the second factor, the evaluation result of the diagnostic result comprises:
determining, based on the first vector, at least one first structured sequence, each of the at least one first structured sequence including a first anatomical structure, a first feature of the first anatomical structure, and a first feature value of the first feature recorded in the diagnostic result;
determining, based on the second vector, at least one second structured sequence, each of the at least one second structured sequence including a second anatomical structure, a second feature of the second anatomical structure, and a second feature value of the second feature recorded in the reference diagnostic result; and
generating, based on the at least one first structured sequence and the second structured sequence, the evaluation result of the diagnostic result.

5. The system of claim 4, wherein the generating, based on the at least one first structured sequence and the second structured sequence, the evaluation result of the diagnostic result further comprises:
generating at least one normalized first structured sequence by normalizing the at least one first structured sequence;
generating at least one normalized second structured sequence by normalizing the at least one second structured sequence; and
generating, based on the at least one normalized first structured sequence and the at least one normalized second structured sequence, the evaluation result of the diagnostic result.

6. The system of claim 4, wherein the generating, based on the at least one first structured sequence and the at least one second structured sequence, the evaluation result of the diagnostic result comprises:
determining first ordering information relating to the diagnostic result, the first ordering information reflecting an order of appearance of the at least one first anatomical structure of the at least one first structured sequence in the diagnostic result;
determining second ordering information relating to the reference diagnostic result, the second ordering information reflecting an order of appearance of the at least one second anatomical structure of the at least one second structured sequence in the reference diagnostic result; and
determining, based on the first ordering information and the second ordering information, a value of an evaluation index relating to a description order of the diagnostic result.

7. The system of claim 6, wherein
the first ordering information further reflects an order of appearance of the at least one first feature of the at least one first anatomical structure in the diagnostic result, and
the second ordering information further reflects an order of appearance of the at least one second feature of the at least one second anatomical structure in the reference diagnostic result.

8. The system of claim 4, wherein the generating, based on the at least one first structured sequence and the at least one second structured sequence, the evaluation result of the diagnostic result comprises:
generating a first comparison result by comparing the at least one first anatomical structure of the at least one first structured sequence and the at least one second anatomical structure of the at least one second structured sequence;
generating a second comparison result by comparing the at least one first feature of the at least one first structured sequence and the at least one second feature of the at least one second structured sequence; and
determining, based on the first compassion result and the second comparison result, a value of an evaluation index relating to a content integrity of the diagnostic result.

9. The system of claim 3, wherein the generating, based on the first vector and the second vector, the evaluation result of the diagnostic result comprises:
determining, based on the first vector, at least one first characteristic value of at least one first lesion specified in the diagnostic result;
determining, based on the second vector, at least one second characteristic value of at least one second lesion specified in the reference diagnostic result; and
determining, based on the at least one first characteristic value and the at least one second characteristic value, a value of an evaluation index relating to an accuracy of the diagnostic result.

10. The system of claim 3, wherein the generating, based on the first vector and the second vector, the evaluation result of the diagnostic result comprises:
determining, based on the first vector, at least one first target vector each of which represents a first element of the diagnostic result;
determining, based on the second vector, at least one second target vector each of which represents a second element of the reference diagnostic result; and
determining, based on the at least one first target vector and the at least one second target vector, a value of an evaluation index relating to a similarity between the diagnostic result and the reference diagnostic result.

11. The system of claim 1, wherein in response to the training request, obtaining a medical image for training the user and a reference diagnostic result with respect to the medical image comprises:
obtaining a user profile of the user;
determining, based on the user profile of the user, a plurality of candidate medical images for training the user; and
selecting, based on a recommendation rule, the medical image from the plurality of candidate medical images.

12. The system of claim 11, wherein the user profile comprises at least one of:
user preference information,
information relating to one or more medical images that have been used to train the user, or
one or more historical diagnostic results inputted by the user.

13. The system of claim 11, wherein the plurality of candidate medical images are determined based on the user profile of the user using a knowledge graph technique.

14. The system of claim 1, wherein in response to the training request, obtaining a medical image for training the user and a reference diagnostic result with respect to the medical image comprises:
generating the reference diagnostic result by evaluating the medical image using a diagnostic result generation model, the diagnostic result generation model being generated using an artificial intelligence technique.

15. A method for medical diagnosis training, comprising:
receiving, from a user terminal, a training request inputted by a user;
in response to the training request, obtaining a medical image for training the user and a reference diagnostic result with respect to the medical image;
transmitting the medical image to the user terminal;
receiving, from the user terminal, a diagnostic result with respect to the medical image inputted by the user; and
generating, based on the reference diagnostic result, an evaluation result of the diagnostic result.

16. The method of claim 15, wherein the generating, based on the reference diagnostic result, an evaluation result of the diagnostic result comprises:
generating a first vector representing the diagnostic result;
generating a second vector representing the reference diagnostic result; and
generating, based on the first vector and the second vector, the evaluation result of the diagnostic result.

17. The method of claim 16, wherein the generating, based on the first vector and the second factor, the evaluation result of the diagnostic result comprises:
determining, based on the first vector, at least one first structured sequence, each of the at least one first structured sequence including a first anatomical structure, a first feature of the first anatomical structure, and a first feature value of the first feature recorded in the diagnostic result;
determining, based on the second vector, at least one second structured sequence, each of the at least one second structured sequence including a second anatomical structure, a second feature of the second anatomical structure, and a second feature value of the second feature recorded in the reference diagnostic result; and
generating, based on the at least one first structured sequence and the second structured sequence, the evaluation result of the diagnostic result.

18. The method of claim 17, wherein the generating, based on the at least one first structured sequence and the at least one second structured sequence, the evaluation result of the diagnostic result comprises:
determining first ordering information relating to the diagnostic result, the first ordering information reflecting an order of appearance of the at least one first anatomical structure of the at least one first structured sequence in the diagnostic result;
determining second ordering information relating to the reference diagnostic result, the second ordering information reflecting an order of appearance of the at least one second anatomical structure of the at least one second structured sequence in the reference diagnostic result; and
determining, based on the first ordering information and the second ordering information, a value of an evaluation index relating to a description order of the diagnostic result.

19. The method of claim 17, wherein the generating, based on the at least one first structured sequence and the at least one second structured sequence, the evaluation result of the diagnostic result comprises:
generating a first comparison result by comparing the at least one first anatomical structure of the at least one first structured sequence and the at least one second anatomical structure of the at least one second structured sequence;
generating a second comparison result by comparing the at least one first feature of the at least one first structured sequence and the at least one second feature of the at least one second structured sequence; and
determining, based on the first compassion result and the second comparison result, a value of an evaluation index relating to a content integrity of the diagnostic result.

20. A non-transitory readable medium, comprising at least one set of instructions, wherein when executed by at least one processor of a system for medical diagnosis training, the at least one set of instructions directs the at least one processor to perform a method, the method comprising:
receiving, from a user terminal, a training request inputted by a user;
in response to the training request, obtaining a medical image for training the user and a reference diagnostic result with respect to the medical image;
transmitting the medical image to the user terminal;
receiving, from the user terminal, a diagnostic result with respect to the medical image inputted by the user; and
generating, based on the reference diagnostic result, an evaluation result of the diagnostic result.
